(19) United States
(12) Patent Application Publication
(10) Pub. No.: US 2021/0214424 A1
(43) Pub. Date: Jul. 15, 2021

(54) NOVEL LEUCINE-RICH REPEAT NEURONAL PROTEIN 1 (LRRN1) ANTIBODIES AND USES THEREOF

(71) Applicants: CHANG GUNG MEMORIAL HOSPITAL, LINKOU, Taoyuan City (TW); ACADEMIA SINICA, Taipei (TW)

(72) Inventors: Alice YU, Taoyuan City (TW); Han-Chung WU, Taipei City (TW); John YU, Taoyuan City (TW)

(21) Appl. No.: 17/054,994

(22) PCT Filed: May 16, 2019
(86) PCT No.: PCT/US19/32675

§ 371 Date: Nov. 12, 2020

Related U.S. Application Data
(60) Provisional application No. 62/672,363, filed on May 16, 2018.

(51) Int. Cl. C07K 16/18; A61K 47/68; A61P 35/00

(52) U.S. Cl. CPC C07K 16/18; A61K 2039/505; A61P 35/00; A61K 47/6843

(57) ABSTRACT

The present invention provides monoclonal antibodies or the antigen-binding portion thereof, that bind to a feto-embryonic dedifferentiated antigen, such as Leucine-rich Repeat Neuronal Protein 1 (LRRN1), a surface glycoprotein that is expressed abundantly on the surface of human embryonic stem cells prior to differentiation into embryoid bodies. Also disclosed herein are the uses of the novel LRRN1 antibodies.

### CROSS-REFERENCE TO RELATED APPLICATIONS

This application claims the benefit of U.S. Patent Application No. 62/672,363, filed on May 16, 2018, the disclosure of which is incorporated by reference herein in their entirety.

### FIELD

The present invention relates to antibodies to Leucine-rich Repeat Neuronal Protein 1 (LRRN1), including specific portions or variants specific for LRRN1, as well as nucleic acids encoding such antibodies, and methods of using thereof, including therapeutic formulations and pharmaceutical compositions comprising the antibody. Further, methods are provided for administering antibodies of the present invention to a subject in an amount effective to inhibit cancer cells or stem cells.

### BACKGROUND

Studies on stem cells, including human embryonic stem cells (hESCs) and cancer stem cells, show that stem cell surface markers are crucial for monitoring the differentiation status or understanding the functional attributes of stem cells. Using glycoproteomics, leucine-rich repeat neuronal protein 1 (LRRN1) was found to be a novel surface marker for human embryonic stem cells (ESCs), which disappears upon differentiation into embryoid bodies (EBs).

LRRN1 is also expressed in many malignance tumor cells, including ovarian cancer, liver cancer, pancreatic cancer, lung cancer, colorectal cancer and breast cancer, see https://www.proteinadas.org/ENSG0000175928-LRRN1/pathology.

These findings support a rationale for the development of antibodies to the stem cell surface glycoprotein, such as LRRN1, as there is still an unmet need for effective treatment and/or prevention for cancer. The present invention provides antibodies to stem cell surface glycoprotein to satisfy these and other needs.

### SUMMARY OF THE INVENTION

The present invention discloses an antibody, or an antigen-binding portion thereof, comprising: (a) a heavy chain variable domain having the amino acid sequence about 90% to 100% identical to the amino acid sequence of SEQ ID NO: 1; and (b) a light chain variable domain having the amino acid sequence about 90 to 100% identical to the amino acid sequence of SEQ ID NO:2.

The present invention also discloses an antibody, or an antigen-binding portion thereof, comprising: (a) a first heavy chain complementarity determining region (HCDR1) having the amino acid sequence of about 90% to 100% identical to SEQ ID NO: 3, SEQ ID NO: 9 or SEQ ID NO: 15, (b) a second heavy chain complementarity determining region (HCDR2) having the amino acid sequence of about 90% to 100% identical to SEQ ID NO: 4, SEQ ID NO: 10 or SEQ ID NO: 16, (c) a third heavy chain complementarity determining region (HCDR3) having the amino acid sequence of about 90% to 100% identical to SEQ ID NO: 5, SEQ ID NO: 11 or SEQ ID NO: 17, (d) a first light chain complementarity determining region (LCDR1) having the amino acid sequence of about 90% to 100% identical to SEQ ID NO: 6, SEQ ID NO: 12 or SEQ ID NO: 18, (e) a second light chain complementarity determining region (LCDR2) having the amino acid sequence of about 90% to 100% identical to SEQ ID NO: 7, SEQ ID NO: 13 or SEQ ID NO: 19, and (f) a third light chain complementarity determining region (LCDR3) having the amino acid sequence of about 90% to 100% identical to SEQ ID NO: 8, SEQ ID NO: 14 or SEQ ID NO: 20.

Also provided are conjugates comprising the antibody or the antigen-binding portion thereof described herein, operatively attached to a therapeutic agent or a diagnostic agent.

Methods to inhibit cancer cells are also provided, by administering the antibody or the antigen-binding portion thereof described herein to a subject in need of thereof.

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various aspects of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification, any or all drawings and each claim.

### BRIEF DESCRIPTION OF THE DRAWINGS

The patent contains at least one drawing executed in color. Copies of this patent with color drawings will be provided by the Office upon request and payment of the necessary fee.

Illustrative embodiments of the present invention are described in detail below with reference to the following Figures.

FIG. 1 is a graphic abstract showing the effect of LRRN1 mediated through AKT on stemness maintenance and mesendoderm differentiation.

FIG. 2A and FIG. 2B are fluorescence microscopy images illustrating the internalization of Alexa Fluor® 488-labeled LRRN1 monoclonal antibody into the breast cancer cells.

FIG. 3 is a bar graph illustrating the antibody-dependent cellular cytotoxicity (ADCC) of LRRN1 E36 monoclonal antibody with or without PBMC incubation, compared to the control, and IgG with or without PBMC incubation.

### DETAILED DESCRIPTION OF THE INVENTION

As used herein, the articles “a” and “an” refer to one or more than one (i.e., at least one) of the grammatical object of the article.

The term “subject” may refer to a vertebrate suspected of having cancer. Subjects include warm-blooded animals, such as mammals, such as a primate, and, more preferably, a human. Non-human primates are subjects as well. The term subject includes domesticated animals, such as cats, dogs, etc., livestock (for example, cattle, horses, pigs, sheep, goats, etc.) and laboratory animals (for example, mouse, rabbit, rat, gerbil, guinea pig, etc.). An “effective amount,” as used herein, refers to a dose of the antibody or conjugate that is sufficient to reduce the symptoms and signs of cancer, such as weight loss, pain and palpable mass, which is detectable, either clinically as a palpable mass or radiologically through various imaging means. The term “effective amount” and “therapeutically effective amount” are used interchangeably.

All numbers herein may be understood as modified by “about.” As used herein, the term “about” is meant to encompass variations of ±10%.

### Antibody

The term “antibody” as used herein refers to immunoglobulin molecules and immunologically active portions of immunoglobulin molecules, i.e., molecules that contain an antigen binding portion that immunospecifically binds a glycoprotein. As such, the term antibody encompasses not only whole antibody molecules, but also antibody fragments as well as variants (including derivatives) of antibodies and antibody fragments. In natural antibodies, two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the stem cell surface glycoprotein. The light and heavy chains of an antibody each have three complementarity determining regions (CDRs), designated LCDR1, LCDR2, LCDR3 and HCDR1, HCDR2, HCDR3, respectively. An antigen-binding site, therefore, includes six CDRs, comprising the CDR set from each of a heavy and a light chain variable region. Framework Regions (FRs) refer to amino acid sequences interposed between CDRs.

Identity or homology with respect to a specified amino acid sequence of this invention is defined herein as the percentage of amino acid residues in a candidate sequence that are identical with the specified residues, after aligning the sequences and introducing gaps, if necessary, to achieve the maximum percent homology or identity, and not considering any conservative substitutions as part of the sequence homology or identity. None of N-terminal, C-terminal or internal extensions, deletions, or insertions into the specified sequence shall be construed as affecting homology or identity. Methods of alignment of sequences for comparison are well known in the art. While such alignments may be done by hand using conventional methods, various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al, Nucleic Acids Research 16:10881, 1988; and Pearson and Lipman, Proc. Natl. Acad. Sci. U.S.A. 85:2444, 1988. Altschul et al., Nature Genet. 6:119, 1994, present a detailed consideration of sequence alignment methods and homology/identity calculations. The NCBI Basic Local Alignment Search Tool (BLAST (Altschul et al, J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity or homology using this program is available on the NCBI website.

Antibodies of the present invention also include chimerized or humanized monoclonal antibodies generated from antibodies of the present invention. In one embodiment, humanized antibodies are antibody molecules from non-human species having one, two or all CDRs from the non-human species and one, two or all three framework regions from a human immunoglobulin molecule. A chimeric antibody is a molecule in which different portions are derived from different animal species. For example, an antibody may contain a variable region derived from a murine mAb and a human immunoglobulin constant region. Chimeric antibodies can be produced by recombinant DNA techniques. Morrison, et al., Proc Natl Acad Sci, 81:6851-6855 (1984). For example, a gene encoding a murine (or other species) antibody molecule is digested with restriction enzymes to remove the region encoding the murine Fc, and the equivalent portion of a gene encoding a human Fc constant region is then substituted into the recombinant DNA molecule. Chimeric antibodies can also be created by recombinant DNA techniques where DNA encoding murine V regions can be ligated to DNA encoding the human constant regions. Better et al., Science, 1988, 240:1041-1043. Liu et al. PNAS, 1987 84:3439-3443. Liu et al., J. Immunol., 1987, 139:3521-3526. Sun et al. PNAS, 1987, 84:214-218. Nishimura et al., Canc. Res., 1987, 47:999-1005. Wood et al. Nature, 1985, 314:446-449. Shaw et al., J. Natl. Cancer Inst., 1988, 80:1553-1559. International Patent Publication Nos. W01987002671 and WO 86/01533. European Patent Application Nos. 184, 187; 171,496; 125,023; and 173,494. U.S. Pat. No. 4,816,567.

Thus, LRRN1 antibodies of the present invention include in combination a heavy chain or light chain variable region, a heavy chain or light chain constant region, a framework region, or any portion thereof, of non-murine origin, preferably of human origin, which can be incorporated into an antibody of the present invention.

Antibodies of the present invention are capable of modulating, decreasing, antagonizing, mitigating, alleviating, blocking, inhibiting, abrogating and/or interfering with at least one LRRN1 expressing cell (e.g., cancer cell or stem cell that expresses LRRN1) activity, such as stemness or differentiation, in vitro, in situ and/or in vivo.

The term “antibody” is further intended to encompass antibodies, digestion fragments, specified portions and variants thereof, including antibody mimetics or comprising portions of antibodies that mimic the structure and/or function of an anti-cancer antibody or specified fragment or portion thereof, including single chain antibodies and fragments thereof, each containing at least one CDR derived from an anti-cancer antibody of the present invention. Functional fragments include antigen-binding portion that bind to LRRN1. For example, antibody fragments capable of binding to LRRN1 or portions thereof, including, but not limited to Fab (e.g., by papain digestion), Fab′ (e.g., by pepsin digestion and partial reduction) and F(ab′)₂ (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments, an isolated CDR, diabodies, triabodies, tetrabodies, linear antibodies, single-chain antibody molecules, and multispecific antibodies formed from antibody fragments are encompassed by the present invenion. Single chain antibodies produced by joining antibody fragments using recombinant methods, or a synthetic linker, are also encompassed by the present invention. Bird et al. Science, 1988, 242:423-426. Huston et al., Proc. Natl. Acad. Sci. USA, 1988, 85:5879-5883.

An antigen-binding portion of an antibody may include a portion of an antibody that specifically binds to a stem cell surface glycoprotein (e.g., LRRN1).

According to one aspect of the invention, the location of the CDRs and framework residues of the VH and VL are determined by one of the following methods: E A Kabat et al. (1991) Sequences of Proteins of Immunological Interest, Fifth Edition, U.S. Department of Health and Human Services, NIH Publication No. 91-3242; MB Swindells et al., “abYsis: Integrated Antibody Sequence and Structure-Management, Analysis, and Prediction.” J Mol Biol. 2017 February 3;429(3):356-364. doi: 10.1016/j.jmb.2016.08.019; J Ye et al., “IgBLAST: an immunoglobulin variable domain sequence analysis tool.” Nucleic Acids Res. 203; 41 (Web server issue): W34-40. doi: 10.1093/nar/gkt38; V Kunik et al., “Structural Consensus among Antibodies Defines the Antigen Binding Site”. PLoS Comput Biol 2012; 8(2): e1002388. doi:10.1371/journal.pcbi.1002388 or V Kunik et al., “Paratome: An online tool for systematic identification of antigen binding regions in antibodies based on sequence or structure.” Nucleic Acids Res. 2012 July;40(Web Server issue):W521-4. doi: 10.1093/nar/gks480. Epub 2012 June 6.

According to another aspect of the invention, the antibody or the antigen-binding portion thereof may have the following structure:

Leader Sequence—FW1-CDR1-FW2-CDR2-FW3-CDR3-

Also encompassed by the present invention are antibodies or antigen-binding portions thereof comprising one or two variable regions as disclosed herein, with the other regions replaced by sequences from at least one different species including, but not limited to, human, rabbits, sheep, dogs, cats, cows, horses, goats, pigs, monkeys, apes, gorillas, chimpanzees, ducks, geese, chickens, amphibians, reptiles and other animals.

The antibodies or antigen-binding portions thereof of the present invention may be monospecific, bi-specific or multispecific. Multispecific or bi-specific antibodies or fragments thereof may be specific for different epitopes of one target stem cell surface glycoprotein (e.g., LRRN1). In one embodiment, a multispecific antibody or antigen-binding portion thereof comprises at least two different variable domains, wherein each variable domain is capable of specifically binding to a separate or to a different epitope on the same stem cell surface glycoprotein. See Tutt et al., 1991, J. Immunol. 147:60-69 and Kufer et al., 2004, Trends Biotechnol. 22:238-244.

All antibody isotypes are encompassed by the present invention, including IgG (e.g., IgG1, IgG2, IgG3, IgG4), IgM, IgA (IgA1, IgA2), IgD or IgE (all classes and subclasses are encompassed by the present invention). The antibodies or antigen-binding portions thereof may be mammalian (e.g., mouse, human) antibodies or antigen-binding portions thereof. The light chains of the antibody may be of kappa or lambda type.

The present invention provides for an antibody, such as a monoclonal antibody, or an antigen-binding portions thereof, comprising a variable region that binds to a stem cell surface glycoprotein (such as LRRN1) or a fragment thereof.

LRRN1 knockdown decreased self-renewal capacity of hESC and skewed differentiation toward endoderm/mesoderm lineages. Mechanistically, silencing of LRRN1 decreases AKT phosphorylation, causes translocation of pluripotency factors OCT4, SOX2 and NANOG from nucleus to cytoplasm which leads to degradation. Thus, LRRN1 is essential for maintaining hESC self-renewal and pluripotency.

In one aspect of the invention, the antibody or the antigen-binding portion thereof comprises a heavy chain variable region, wherein the heavy chain variable region comprises three CDRs, i.e., HCDR1, HCDR2 and HCDR3, wherein HCDR1 having amino acid sequences about 90%, about 91%, about 92%, about 93%, about 94%, about 95%, about 96%, about 97%, about 98%, about 99% or about 100% homologous or identical to the amino acid sequence set forth in SEQ ID NOs: 3, 9 or 15, HCDR2 having amino acid sequences about 90%, about 91%, about 92%, about 93%, about 94%, about 95%, about 96%, about 97%, about 98%, about 99% or about 100% homologous or identical to the amino acid sequence set forth in SEQ ID NOs: 4, 10 or 16, HCDR3 having amino acid sequences about 90%, about 91%, about 92%, about 93%, about 94%, about 95%, about 96%, about 97%, about 98%, about 99% or about 100% homologous or identical to the amino acid sequence set forth in SEQ ID NOs: 5, 11 or 17.

In another aspect of the invention, the antibody or the antigen-binding portion thereof comprises a light chain variable region, wherein the light chain variable region comprises three complementarity determining regions (CDRs), i.e., LCDR1, LCDR2 and LCDR3, wherein LCDR1 having amino acid sequences about 90%, about 91%, about 92%, about 93%, about 94%, about 95%, about 96%, about 97%, about 98%, about 99% or about 100% homologous or identical to the amino acid sequence set forth in SEQ ID NOs: 6, 12, or 18, LCDR2 having amino acid sequences about 90%, about 91%, about 92%, about 93%, about 94%, about 95%, about 96%, about 97%, about 98%, about 99% or about 100% homologous or identical to the amino acid sequence set forth in SEQ ID NOs: 7, 13 or 19, LCDR3 having amino acid sequences about 90%, about 91%, about 92%, about 93%, about 94%, about 95%, about 96%, about 97%, about 98%, about 99% or about 100% homologous or identical to the amino acid sequence set forth in SEQ ID NOs: 8, 14 or 20.

In one embodiment, the antibody or the antigen-binding portion thereof comprises a heavy chain variable region (VH) having an amino acid sequence about 90%, about 91%, about 92%, about 93%, about 94%, about 95%, about 96%, about 97%, about 98%, about 99% or about 100% homologous or identical to SEQ ID NO: 1, and/or a light chain variable region (VL) comprises a light chain having an amino acid sequence about 90%, about 91%, about 92%, about 93%, about 94%, about 95%, about 96%, about 97%, about 98%, about 99% or about 100% homologous or identical to SEQ ID NO:2.

Table 1 shows the amino acid sequences of the heavy chain variable region, the light chain variable region, and the CDRs of one embodiment of the antibody of the present invention.

TABLE 1
Amino Acid Sequence of the LRRN1 antibody
of the present invention

| | | SEQ |
|---|---|---|
| Chain | | ID |
| Region | Amino Acid Sequence | NO. |
| Heavy | QVQLQESGGGLVQPGGSLRLSCTSSGLSISDR | 1 |
| Chain | WMSWVRQAPGKKLEWVADIEHDGRAKYYADSV | |
| Variable | RGRFTISRDNAQNSVYLQMSSLRAEDTAVYFC | |
| Region | ATWAGPNWRMDYWGQGTLVTVSS | |
| (VH) | | |
| Light | ETTLTQSPGTLSLSPGERATLSCRASQSVTSS | 2 |
| Chain | YLAWYQQKPGQAPRLLIYGASSRATGIPDRFS | |
| Variable | GSGSGTDFTLTISRLEPEDFAVYYCQQYGNSP | |
| Region | LTFGGGTKVEIKR | |
| (VL) | | |
| HCDR1¹ | GLSISDR | 3 |
| HCDR2¹ | EHDGRA | 4 |
| HCDR3¹ | WAGPNWRMDY | 5 |
| LCDR1¹ | RASQSVTSSYLA | 6 |
| LCDR2¹ | GASSRAT | 7 |
| LCDR3¹ | QQYGNSPLT | 8 |
| HCDR1² | GLSISDRW | 9 |
| HCDR2² | IEHDGRAK | 10 |
| HCDR3² | ATWAGPNWRMDY | 11 |
| LCDR1² | QSVTSSY | 12 |
| LCDR2² | GAS | 13 |
| LCDR3² | QQYGNSPLT | 14 |
| HCDR1^{3and4} | LSISDRWMS | 15 |
| HCDR2^{3and4} | WVADIEHDGRAKYY | 16 |
| HCDR3^{3and4} | TWAGPNWRMDY | 17 |
| LCDR1^{3and4} | QSVTSSYLA | 18 |
| LCDR2^{3and4} | LLIYGASSRAT | 19 |
| LCDR3^{3and4} | QQYGNSPL | 20 |

¹The sequence was determined according to Swindells MB, Porter CT, Couch M, et al., (2017) abYsis: Integrated Antibody Sequence and Structure-Management, Analysis, and Prediction. J Mol Biol. 2017 Feb. 3;429(3):356-364. doi: 10.1016/j.jmb.2016.08.019.
²The sequence was determined according to Ye J, Ma N, Madden TL and Ostell JM (2013). IgBLAST: an immunoglobulin variable domain sequence analysis tool. Nucleic Acids Res. 41 (Web server issue): W34-40. doi: 10.1093/nar/gkt382
³The sequence was determined according to Kunik V, Peters B, Ofran Y (2012). Structural Consensus among Antibodies Defines the Antigen Binding Site. PLoS Comput Biol 8(2): e1002388. doi:10.1371/journal.pcbi.1002388.
⁴The sequence was determined according to Kunik V, Ashkenazi S, Ofran Y (2012). Paratome: An online tool for systematic identification of antigen binding regions in antibodies based on sequence or structure. Nucleic Acids Res. 2012 July; 40(Web Server issue):W521-4. doi: 10.1093/nar/gks480. Epub 2012 Jun. 6

In one embodiment, the antibody or antigen-binding portion thereof binds to a stem cell surface glycoprotein, including but not limited to LRRN1.

In one embodiment, the antibody or antigen-binding portion thereof have in vitro and in vivo therapeutic, prophylactic, and/or diagnostic utilities. For example, these antibodies can be administered to cells in culture, e.g., in vitro or ex vivo, or to a subject, e.g., in vivo, to treat, inhibit, prevent relapse, and/or diagnose diseases, such as cancer.

### Methods for Inhibiting Cancer Cells or Human Stem Cells

Antibodies or conjugates of the present invention are capable of modulating, decreasing, antagonizing, mitigating, alleviating, blocking, inhibiting, abrogating and/or interfering with at least one stem cell surface glycoprotein or a fragment thereof in vitro, in situ and/or in vivo.

The invention also provides methods for inhibiting the growth of a cancer cell or a stem cell in vitro, ex vivo or in vivo, wherein the cancer cell or the stem cell is contacted with an effective amount of an antibody or the conjugate as described herein. The cancer cells and stem cells express a stem cell surface glycoprotein, such as LRRN1. Non limiting examples of LRRN1 expressing cancer include glioma, lymphoma, lung cancer, pancreatic cancer, carcinoid, colorectal cancer, head and neck cancer, gastric cancer renal cancer, urothelial cancer, testis cancer, cervical cancer, ovarian cancer, endometrial cancer, breast cancer, skin cancer or melanoma.

In vitro efficacy of the present antibody or the conjugate can be determined using methods well known in the art. MTT assay is based on the principle of uptake of MTT, a tetrazolium salt, by metabolically active cells where it is metabolized into a blue colored formazan product, which can be read spectrometrically. J. of Immunological Methods 65: 55 63, 1983. The cytotoxicity of the present antibody or the antigen-binding portion thereof may be studied by colony formation assay. Functional assays for binding LRRN1 may be performed via ELISA. Cell cycle block by the antibody or the antigen-binding portion thereof may be studied by standard propidium iodide (PI) staining and flow cytometry. Invasion inhibition may be studied by Boyden chambers. In this assay a layer of reconstituted basement membrane, Matrigel, is coated onto chemotaxis filters and acts as a barrier to the migration of cells in the Boyden chambers. Only cells with invasive capacity can cross the Matrigel barrier. Other assays include, but are not limited to cell viability assays, apoptosis assays, and morphological assays. Assays can also be done in vivo using a murine model. See, e.g., B. Teicher, Tumor Models for Efficacy Determination. Mol Cancer Ther 2006; 5: 2435-2443.”

### Conjugate

In some embodiments, the antibodies or the antigen-binding portion thereof can be linked to or co-expressed with another functional molecule, e.g., a diagnostic agent or a therapeutic agent, to form a conjugate. For example, an antibody or the antigen biding portion thereof can be operatively attached to (e.g., by chemical coupling, genetic fusion, recombinant expression, a cleavable spacer or linker, covalent or noncovalent association or otherwise) one or more other molecular entities.

In one embodiment, the therapeutic agent can enhance and even synergise the effects of the antibody of the present invention. Non limiting examples of the therapeutic agent include chemotherapeutic agents, anti-angiogenic agents, apoptosis-inducing agents and anti-tubulin drugs or a second monoclonal antibody or polyclonal antibody.

Exemplary chemotherapeutic agents include: steroids; cytokines; anti-metabolites, such as cytosine arabinoside, fluorouracil, methotrexate or aminopterin; anthracyclines; mitomycin C; vinca alkaloids; antibiotics; demecolcine; etoposide; mithramycin; and anti-tumor alkylating agents, such as chlorambucil or melphalan. Exemplary anti-angiogenic agents include angiostatin, endostatin, vasculostatin, canstatin and maspin. Exemplary anti-tubulin drugs include colchicine, taxol, vinblastine, vincristine, vindesine and combretastatins (e.g., combretastatin A, B and/or D). Exemplary anti-tubulin drugs are colchicine; taxanes, such as taxol; vinca alkaloids, such as vinblastine, vincristine and vindesine; and combretastatins.

The following therapeutic agents have been reported to conjugate to an antibody: doxorubicin, daunomycin, methotrexate and vinblastine neocarzinostatin, macromycin, trenimon and a-amanitin (see U.S. Pat. Nos. 5,660,827; 5,855,866; and 5,965,132).

Routes of administration of the present antibody and conjugate include, but are not limited to, intravenous, intramuscular, intranasal, subcutaneous, oral, topical, intradermal, transdermal, subdermal, parenteral, rectal, spinal, or epidermal administration.

The antibody or conjugate can be administered in a single dose treatment or in multiple dose treatments on a schedule and over a time period appropriate to the age, weight and condition of the subject, the particular composition used, and the route of administration, for prophylactic or curative purposes, etc. For example, in one embodiment, the antibody or the conjugate according to the invention is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid).

For ease of administration and uniformity of dosage, oral or parenteral dosage unit form may be used. Dosage unit form as used herein refers to physically discrete units suited as unitary dosages for the subj ect to be treated; each unit containing a predetermined quantity of antibody calculated to produce the desired therapeutic effect.

The data obtained from the cell culture assays and animal studies can be used in formulating a range of dosage for use in humans. In one embodiment, the dosage of the antibody or conjugate lies within a range of circulating concentrations that include the ED₅₀ with little or no toxicity. The dosage can vary within this range depending upon the dosage form employed and the route of administration utilized. In another embodiment, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Sonderstrup, Springer, Sem. Immunopathol. 25: 35-45, 2003. Nikula et al., Inhal. Toxicol. 4(12): 123-53, 2000.

The effective amount of the antibody or the conjugate depends on the subject and the condition to be treated. In one embodiment, the present antibody or antigen-binding portion thereof is administered at a dose ranging from about 0.01 mg to about 10 g, from about 0.1 mg to about 9 g, from about 1 mg to about 8 g, from about 2 mg to about 7 g, from about 3 mg to about 6 g, from about 10 mg to about 5 g, from about 20 mg to about 1 g, from about 50 mg to about 800 mg, from about 100 mg to about 500 mg, from about 0.01 μg to about 10 g, from about 0.05 μg to about 1.5 mg, from about 10 μg to about 1 mg protein, from about 30 μg to about 500 μg, from about 40 μg to about 300 μg, from about 0.1 μg to about 200 μg, from about 0.1 μg to about 5 μg, from about 5 μg to about 10 μg, from about 10 μg to about 25 μg, from about 25 μg to about 50 μg, from about 50 μg to about 100 μg, from about 100 μg to about 500 μg, from about 500 μg to about 1 mg, from about 1 mg to about 2 mg. The specific dose level for any particular subject depends upon a variety of factors including the activity of the specific peptide, the age, body weight, general health, sex, diet, time of administration, route of administration, and rate of excretion, drug combination and the severity of the particular disease undergoing therapy and can be determined by one of ordinary skill in the art without undue experimentation.

The following examples of specific aspects for carrying out the present invention are offered for illustrative purposes only, and are not intended to limit the scope of the present invention in any way.

### EXAMPLES

### Materials and Methods

### Cell Culture and Reagents

The hESC cell lines HES-5 and H9 were obtained from ES Cell International (ESI, Singapore) and WiCell Research Institute (USA), and were cultured on irradiated mouse embryonic fibroblast (MEF) layers in Matrigel-coated plates. The hESC growth medium consisted of 80% DMEM/F12, 20% Knockout Serum Replacement (Invitrogen, USA), 1 mM L-glutamine, 0.1 mM β-mercaptoethanol and 4 ng/mL FGF-2 (Invitrogen). Before use, the hESC growth medium was conditioned on mitomycin-C (Sigma-Aldrich, USA) inactivated MEFs for 24 h at a density of 1.2×10⁵ cells/mL.

To induce differentiation of hESCs into EBs, hESCs were treated with 1 mg/mL dispase for approximately 30 min, until the cells had completely detached from the plates. Then, the cell suspensions were transferred to conical tubes. After the cells had settled by gravity, the medium was removed and the cells were washed twice with hESC growth medium. To induce EB outgrowth, cells were transferred to an ultra-low-attachment cell culture flask (Corning) containing hESC growth medium, without FGF-2, for approximately 48 h. Then, the samples were grown on gelatin coated tissue culture dishes or flasks with culture medium consisting of 80% DMEM/F12, 20% FBS, 1 mM L-glutamine, 0.1 mM β-mercaptoethanol, and 0.5% penicillin and streptomycin for another 2 weeks. The medium was changed every other day.

### Plasmid Production and Lentiviral Transduction

For plasmid construction, full-length coding sequences of human LRRN1 (LRRN1, accession number NM 020873.5, SEQ ID NO:21) were cloned with a specific forward primer (GATCGGATCCATGGCTAGGATGAGCTTTGTTATAGC A, SEQ ID NO: 22) and a reverse primer (GATCCTCGAGTTACCACATGTAATAG CTTCTGGATGTGT, SEQ ID NO: 23) into pLKO AS3W.puro vector (National RNAi Core Facility). Cells were infected by viruses at a multiplicity of infection (MOI) of 10 with the addition of 8 μg/mL polybrene (Sigma-Aldrich).

### Quantitative RT-PCR

Total RNA was extracted from hESCs and EB outgrowth cells, using an RNeasy Mini Kit, and treated with an RNase-free DNase I set (Qiagen, Hilden, Germany) according to the manufacturer's protocol. Total RNA (1 μg) was reverse-transcribed using oligo (dT)₁₅ primers and a reverse transcription system (Promega). Quantitative RT-PCR (qPCR) was carried out using LightCycler 480 SYBR Green I Master mix (Roche) and analyzed with a LightCycler 480 II real-time PCR system (Roche). The following marker genes were studied to differentiate trophectoderm, endoderm, mesoderm and ectoderm: GAPDH, LRRNJ, OCT4, NANOG, SOX2, CDX2, EOMES, HAND1, GATA6, GATA4, SOX17, AFP, Brachyury, WTI, TWIST, BMP4, SOX1, PAX6 and NEUROD1. GAPDH was used as an internal control.

### Western Blotting

Cell extracts were prepared from cells that were suspended in RIPA lysis buffer with protease inhibitor (Roche, Switzerland). After centrifugation, supernatants were dissolved in the Laemmli sample buffer (Bio-Rad, USA) for sodium dodecyl sulfate-polyacrylamide gel electrophoresis (SDS-PAGE). Approximately 50 μg of protein were separated in SDS-PAGE and electrotransferred onto a PVDF membrane. The membrane was blocked with 5% skim milk and then probed with the following primary antibodies: anti-LRRN1 (AF4990, R&D Systems, USA), anti-OCT4 (sc-5279, Santa Cruz Biotechnology, USA), anti-NANOG (ab21624, Abcam, UK), anti-SOX2 (MAB4343, Merck Millipore, USA), anti-phosphorylated AKT (4060, Cell Signaling Technology, USA), anti-AKT (sc-8312, Santa Cruz) and anti-ACTIN (A5441, Sigma-Aldrich) at 4 degree for overnight. After incubation with horseradish peroxidase-conjugated secondary antibody (Jackson ImmunoResearch, USA), the membrane was then visualized using Immobilon Western Chemiluminescent HRP Substrate (Millipore). The Western blotting results were quantified with Image-Quant 5.2 software (GE Healthcare, USA).

The half-lives of OCT4, NANOG and SOX2 proteins were calculated from the slope of the semi-log transformed best-fit lines. The decay curves were analyzed individually using linear regression of protein amount, and expressed as a percentage of protein remaining vs. time, as previously described in O. Adewumi et al., Characterization of human embryonic stem cell lines by the International Stem Cell Initiative. Nature biotechnology. 2007;25:803.

### Protein Stability Assay

To assay the stability of OCT4, NANOG and SOX2, the expression of LRRN1 in hESCs was silenced using a lentiviral plasmid (available from National RNAi Core Facility of the Institute of Molecular Biology, Academia Sinica, Taiwan). The LRRN1 silenced cells and controlled cells were treated with 100 μg/mL of cycloheximide (Millipore) to inhibit protein synthesis and subsequently harvested after 0, 30, 60, 90, and 120 min. To examine the decay of OCT4, NANOG, and SOX2 by proteasome degradation, LRRN1 silenced hESCs were pretreated with 10 μM carbobenzoxyl-Leu-Leu-leucinal (MG132) (Sigma-Aldrich) to inhibit proteasome activity for 2 h prior to the incubation with CHX. Then, cells were harvested at 30, 60, 90, and 120 min and the samples were processed for Western blot analysis.

### Cell Proliferation Assay

Cell proliferation was determined with the LEAP® (Laser-Enabled Analysis and Processing) Cell Processing Workstation (Intrexon Corp, USA) according to the manufacturer's instructions. Briefly, controlled cells and LRRN1 silenced hESCs were seeded at 5×10³ cells per well in a 96 well plate. Then, cells were separately stained with DAPI (Sigma-Aldrich) on days 1 to 3 and were visualized using the LEAP®. Growth curves for control and LRRN1 silenced hESCs were measured on days 1, 2, and 3, using the LEAP® Workstation to determine cell numbers from DAPI stained nuclei.

### Cell Viability Determination

Cell viability was determined by incubation with 10% AlamarBlue reagent (Biosource International, USA) for 2 h, followed by fluorescence measurement (excitation: 544 nm, emission: 590 nm) using a spectrophotometer (Spectramax 190, Molecular Devices, USA).

### Flow Cytometry and Confocal Microscopic Analysis

To examine the surface expression of LRRN1, flow cytometry analysis was performed with anti-LRRN1 (E36) monoclonal antibodies. Single cell suspension of hESCs (H9) and 13-day EBs were prepared and co-incubated with LRRN1 E36 monoclonal antibody of the present invention at 4° C. for 60 mins. After washing with cold PBS, secondary antibody-conjugated with Alexa Fluor 488 (Jackson ImmunoResearch) and 7-AAD (BD Biosciences) were added at 4° C. for 20 mins. The samples were washed and assayed with flow cytometer (EC-800 instrument, Sony Biotecnology, USA). Data analysis was performed with FlowJo software. Percentage of anti-LRRN1⁺ cells was analyzed after gating live cell population (i.e., 7-AAD⁻ cells).

For confocal imaging analysis, hESCs were seeded on chamber slide (Ibidi), grown as described above, washed with PBS, fixed with 4% paraformaldehyde/PBS for 15 min at 4° C., rinsed in PBS, and blocked with 5% BSA/PBS for 30 min at room temperature. Sample were incubated with IgG control and LRRN1 E36 monoclonal antibody (1:100) for 1 hour at room temperature. Then, cells were washed with PBS and incubated with Alexa Fluor 488 AffiniPure goat anti-human IgG secondary antibody (Jackson ImmunoResearch) for 1 hour at room temperature, and counterstained with DAPI. The intensity and localization of LRRN1 staining was monitored by confocal microscopy (LEICA TCS SP8); DAPI and Alexa-488 images were collected with a 100×/1.4 oil immersion objective in combination with a Hybrid detector. The resulting z-stacked fluorescent images were analyzed using LAS X software (LEICA).

### Immunofluorescence Analysis

After transduction of hESCs for 7 days, the controlled hESCs and LRRN1 silenced hESCs (using a lentivirus plasmid) were fixed in 4% paraformaldehyde/phosphate-buffered saline (PBS) for 15 min at room temperature, permeabilized with 0.5% Triton X-100 in PBS for 5 min, and then cells were blocked with 5% bovine serum albumin (BSA)/PBS for 30 min. Then the cells were incubated at 4° C. with primary antibodies. For germ layer marker analysis, the following primary antibodies were used: anti-HAND1 (sc-9413, Santa Cruz), anti-SOX17 (AF1924, R&D), anti-a-fetoprotein (ab21624, Abcam), anti-WT1 (sc-192, Santa Cruz) and anti-PAX6 (sc-53106, Santa Cruz). For nuclear location analysis, primary antibodies recognized OCT4 (Santa Cruz), NANOG (Abcam), and SOX2 (Millipore) were used.

After overnight incubation with primary antibodies, cells were washed three times with PBS and incubated for 1 h at room temperature with secondary antibodies. The secondary antibodies used were Alexa Fluor 488-conjugated goat anti-mouse and anti-rabbit IgG, and Alexa Fluor 555-conjugated goat anti-mouse and anti-rabbit IgG (Invitrogen). After washing with PBS, cells were incubated with DAPI for nuclear staining and then visualized by fluorescence microscopy (LEICA DMI3000B). To block the nuclear export of OCT4, NANOG and SOX2, LRRN1 silenced hESCs were treated with MG132 for 2 h and then with 20 ng/mL leptomycin B (LMB, Sigma-Aldrich) for another 6 h in the presence of MG132.

### Teratoma Formation

Approximately 2-5×10⁶ hESCs or LRRN1 silenced hESCs (using a lentirus plasmid, Academia Sinica) were resuspended in 200 μL of Hank's Balanced Salt Solution and subcutaneously injected into five-week old NOD/SCID mice. After 8 weeks, controlled hESCs and LRRN1 silenced hESCs formed teratomas were surgically collected, and then fixed with 4% formaldehyde, and paraffin embedded. Immunohistochemical analysis was performed to identify the three embryonic germ layers. For labeling with antibodies against the three germ layer markers SOX17 (endoderm marker), aSMA (mesoderm marker) and NESTIN (ectoderm marker), the general immunohistochemical staining protocol was performed.

### Data Mining Using Stemformics Database

The relative expression of LRRN1 mRNA in undifferentiated hESCs or iPSCs, as compared with their various differentiated derivatives were analyzed using Stemformatics (www.stemformatics.or) database. Log2 expression of LRRN1 results were collated from different datasets, and assessed for differential expression of LRRN1. Datasets containing both stem cells and differentiated cells were chosen, and the result read out from disease sample was excluded and wild type or healthy donor cells were collected. Datasets used are Maherali_2008_18786420, Guenther_2010_20682450, Marchetto_2010_21074045, Jia_2010_20139967, Hu_2011_21296996, Zhang_2011_21185252, Koyanagi-Aoi_2013_24259714a, and Petrova_2014_24936454.

### RESULTS

LRRN1 is Highly Expressed in hESCs

Glycoproteomic analysis was used to compare the glycoprotein expression patterns of undifferentiated hESCs (HES-5) and 16-day EB outgrowth cells, by (i) incubation with ManNAcyne and incorporation of ManNAcyne into sialylated proteins by glycan biosynthetic machinery in hESCs and EB outgrowth cells, (ii) cell lysis and click chemistry reactions to link the alkynyl sialylated glycoconjugates and biotin azide, (iii) affinity capture of the biotin-labeled sialylated glycoconjugates with streptavidin agarose beads, (iv) trypsin digestion of the sialylated glycoproteins on the beads followed by digestion with PNGase F, and (v) LC-MS/MS analysis of the peptide mixture, which identified N-linked sialoglycoproteins that are enriched in hESCs. The glycoproteomic analysis showed ALPL, PROM1, THY1 and LRRN1 have greater expression in hESCs, as compared with EB.

To examine the cellular localization of LRRN1, flow cytometric and confocal microscopic analysis were performed using intact cells. The LRRN1 E36 monoclonal antibody specifically recognized LRRN1 (see Table 1 for the amino acid sequences) was used in flow cytometric analysis of intact cells.

LRRN1 E36 monoclonal antibody was generated from a known phage displayed scFv antibody libraries technology platform to isolate the targeting ligands which specifically bind to plasma membrane markers of human embryonic stem cells (hESCs). Specific phage clones have been identified, which could bind to the undifferentiated human embryonic stem cells and monitored stages of stem cell differentiation and development according to expression levels of these surface markers. Furthermore, specific targeting ligands was used for undifferentiated hESCs to purify, characterize and undertake a functional study of the target proteins on stem cells (e.g. LRRN1). Then, the sequences of single clones of phage particles bearing the target ligand were determined; and the DNA inserted into plasmid and produced in 293T cells for mAb production. The epitope of LRRN 1 E36 antibody was found to be located at LRR domain of LRRN1.

90.7% of hESCs and 11.8% of EB outgrowth cells were positive for LRRN1 expression, determined using LRRN1 E36 monoclonal antibody. Confocal microscopy of immunostained undifferentiated hESCs showed that LRRN1 is localized at the surface of the cell. Furthermore, mRNA level of LRRN1 was at least 4-fold higher in hESCs cells, as compared to EB outgrowth cells. The protein level of LRRN1 was 4- to 10-fold higher in both HES-5 and H9 hESCs, compared to EB outgrowth cells.

The LRRN1 expression in hESC prior to the induction of differentiation was significantly higher than that in differentiated derivatives (20-800 fold in various datasets). The mRNA level of LRRN1 was greater expressed in iPSC (430±111 fold increase) compared with that in fibroblast. The mRNA levels of LRRN1 decreased to 13.5±8.9 after differentiation of iPSC. Overall, these data confirm that the expression of LRRN1 could be a unique marker for undifferentiated hESC.

The Effect of LRRN1 Knockdown in hESCs

LRRN1 expression was silenced in hESCs using a lentivirus plasmid (Academia Sinica) for 3 days, the protein expression of LRRN1 was knocked down by about 90% (relative fold 1.0 to 0.1). LRRN1 knocked down led to an obvious decrease in proliferation of hESCs, due to increased apoptosis after 7 days of cultures.

The Effect of LRRN1 Silencing in hESCs

mRNA levels of the three germ layer markers in control hESCs and LRRN1-silenced hESCs (using a lentivirus plasmid, available from Academia Sinica) were measured by qPCR at day 7. EOMES, GATA4, α-fetoprotein (AFP), SOX17, Brachyury, WTI and TWIST were upregulated at least 4-fold in LRRN1-silenced cells compared with control hESCs. In contrast, LRRN1 silencing had little effect on the levels of SOX1, PAX6 and NEUROD1. These results imply that LRRN1 is essential for the maintenance of pluripotency of hESCs. PAX6 expression was not observed by immunofluorescence staining, suggesting the lack of differentiation toward ectoderm lineage after LRRN1 silencing).

In the in vivo teratoma formation assay, hESCs could differentiate into three germ layers. However, only endoderm and mesoderm were observed in LRRN1-silenced hESCs. Therefore, loss-of-function for LRRN1 in hESCs resulted in a developmental skewing toward endoderm and mesoderm lineages in vitro and in teratoma assays.

Gene expression profiles were analysed using qPCR and the mRNA levels of OCT4, NANOG, and SOX2 did not show significant differences with or without LRRN1 silencing in hESCs. Overall, silencing of LRRN1 in hESCs did not influence the mRNA levels for the pluripotency factors, but somehow affected the differentiation capacity of hESCs.

### The Effect of LRRN1 on Pluripotency Factors

The levels of NANOG and SOX2 proteins in LRRN1 silenced cells were reduced to 30% and 20% of control values, respectively, as shown by western blot analysis post 5 days infection. In contrast, OCT4 protein expression was only slightly reduced (70% of control).

The control- and LRRN1 silenced hESCs were treated with cycloheximide (CHX), which inhibits new protein synthesis, followed by western blot analysis to examine the stability of endogenous OCT4, NANOG, and SOX2. The cells were harvested after CHX treatment, which showed OCT4, NANOG and SOX2 protein levels were reduced within 2 h of CHX treatment in both control and LRRN1-silenced hESCs. In control cells, OCT4, NANOG and SOX2 exhibited half-lives of 118, 69, and 58 min, respectively. However, in LRRN1-silenced cells, OCT4, NANOG and SOX2 had shortened half-lives of 71, 35, and 21 min.

The stabilities of OCT4, NANOG and SOX2 in hESCs in the presence of MG132 (a proteasome inhibitor) was assessed to determine if the instability of OCT4, NANOG and SOX2 after silencing of LRRN1 could be attributed to the proteasome pathway. LRRN1 silenced cells were pretreated with MG132 for 2 h before CHX inoculation. The cells were then harvested and analyzed for OCT4, NANOG and SOX2 by western blot analysis. The addition of MG132 increased the expression levels and half-lives of OCT4, NANOG and SOX2 caused by LRRN1 silencing. These results suggest reduced LRRN1 expression suppresses OCT4, NANOG and SOX2 expression by activating proteasomal degradation.

The effect of LRRN1 silencing on the translocation of the OCT4, NANOG and SOX2 proteins in hESCs was examined, using immunofluorescence staining. In control cells, OCT4, NANOG and SOX2 proteins were predominantly localized in the nucleus and not in cytoplasm whereas after silencing LRRN1, these proteins exhibited dramatically less nuclear green fluorescence, but no fluorescence was observed in the cytoplasm. Remarkably, MG132 treatment increased the percentage of cells with OCT4 (36%), NANOG (32%) and SOX2 (42%) in the cytoplasm. Furthermore, treatment with LMB, a specific inhibitor of the nuclear export receptor CRM1, decreased the percentage of cells with OCT4 (6%), NANOG (8%) and SOX2 (10%) in the cytoplasm. The observation that LMB treatment reversed the effect of LRRN1 silencing, suggests that the translocation of nuclear OCT4, NANOG and SOX2 to the cytoplasm is at least partially dependent on the CRM1-mediated nuclear export pathway.

Given silencing of LRRN1 shortened the half-lives of the pluripotency factors (OCT4, NANOG and SOX2), the role of AKT signaling to affect the stability of the pluripotency factors was examined. LRRN1-silenced cells showed significantly lower levels of the phosphorylations of AKT. Both p-AKT (S473) and p-AKT (T308) levels were reduced to 48% and 35% compared to that of controls, respectively.

Since basic fibroblast growth factor (bFGF) is a key activator of AKT signaling, the possibility of LRRN1 overexpression to compensate for the decreased AKT signaling when bFGF was removed from the growth medium was investigated. hESCs were transfected for 24 h with lentiviruses expressing the full-length LRRN1, resulting in LRRN1 overexpression and cultured in growth medium without bFGF for another 24 or 48 h. At 0 h after the removal of bFGF, p-AKT (S473) protein levels in control cells and LRRN1 overexpressing cells were similar. However, after withdrawal of bFGF for 24 or 48 h, the levels of p-AKT (S473) in control cells decreased in a time-dependent manner. In contrast, overexpression of LRRN1 prevented the decline in p-AKT (S473) caused by the absence of bFGF.

The effect of LRRN1 overexpression on the translocation of OCT4, NANOG and SOX2 from nucleus to cytoplasm in the hESCs in the absence of bFGF stimulation was examined. In control cells with bFGF, fluorescent labeled OCT4, NANOG and SOX2 proteins were all observed in the nucleus. After removing bFGF from the control cells, labeled OCT4, NANOG, and SOX2 proteins showed less nuclear fluorescence. However, LRRN1 overexpression in hESCs without bFGF dramatically restored nuclear fluorescent labeling of OCT4, NANOG and SOX2. These data suggest LRRN1 activates AKT in hESCs and this activation may affect the subcellular distribution of pluripotency factors.

The effect of AKT inhibition and LRRN1 silencing on the expression of pluripotency factors was studied using a specific AKT inhibitor (AKTi-1/2). The result shows AKTi-1/2 treatment did not affect the viability of hESCs at concentrations up to 40 μM and there was a dose-dependent decrease in OCT4 and SOX2 protein levels concomitant with the decreases in pAKT level. In contrast, the NANOG protein level was only slightly reduced with AKTi-1/2 concentrations up to 20 μM, but declined dramatically with increased AKTi-1/2 concentration up to 40 uM. Immunostaining was performed on hESCs after AKTi-1/2 treatment in the presence or absence of MG132 and LMB. In untreated cells, OCT4, NANOG, and SOX2 proteins all accumulated in the nucleus whereas after AKTi-1/2 treatment, OCT4 and SOX2 labeling exhibited less intense nuclear fluorescence. NANOG labeling displayed more intense fluorescence in the nucleus. MG132 treatment dramatically increased percentages of cells with fluorescence from OCT4 (from 0 to 49%), NANOG (from 0 to 45%) and SOX2 (from 0 to 48%) in the cytoplasm of AKTi-1/2 treated cells.

The localization of the pluripotency factors after the addition of LMB to cells pretreated with AKTi-1/2 and MG132 was assessed. The result shows blocking nuclear export reversed the effect of MG132 and decreased the percentage of cells with fluorescence due to OCT4 (from 31% to 8%), NANOG (from 45% to 5%) and SOX2 (from 48% to 0) in the cytoplasm.

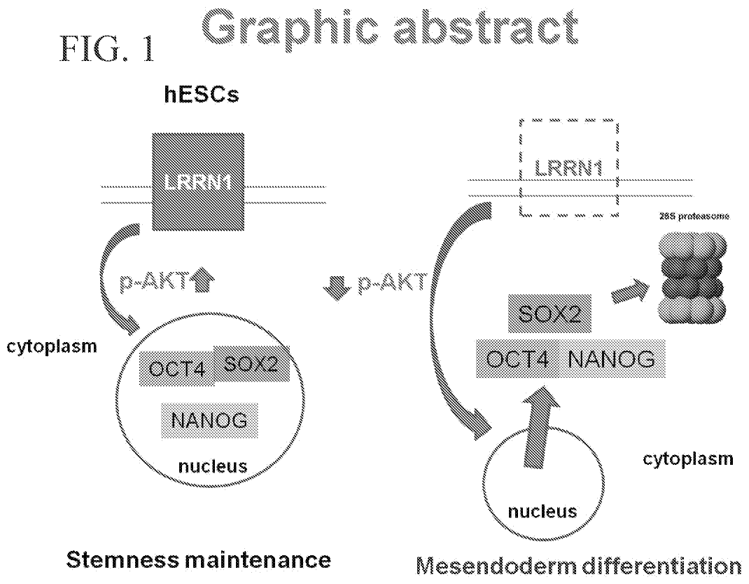

These results indicate that phosphorylation of AKT plays an important role in the subcellular distribution of the OCT4, NANOG and SOX2 and the effect of LRRN1 silencing on nuclear translocation and proteolysis of pluripotency proteins may be mediated through AKT suppression, as illustrated in FIG. 1.

### Antibody-Drug Conjugate

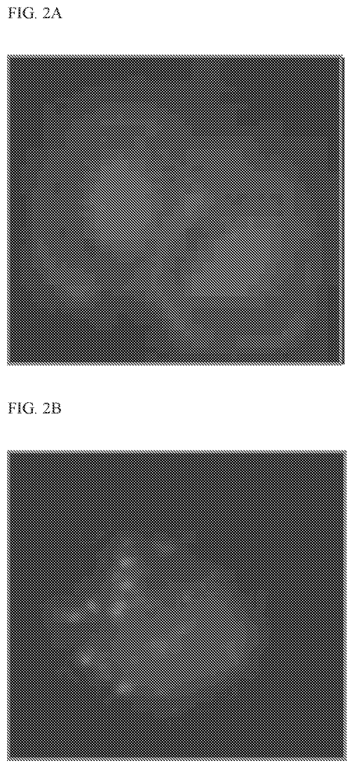

To verify LRRN1 E36 monoclonal antibody of the present inventioncan be used as an antibody-drug conjugate (ADC) vehicle, an antibody internalization assay was carried out using fluorescence microscopy in MCF7 breast cancer cells. The breast cancer cells were stained with LRRN1 E36 monoclonal antibody on ice for 1 hour and then incorporated with goat anti-human IgG conjugated alexa fluor-488 on ice for another 1 hour. Afterward, the LRRN1 E36 monoclonal antibody-treated breast cancer cells were incubated at 4° C. or 37° C. for 3 hours. Referring to FIG. 2A and FIG. 2B, immunofluorescence analysis shows the internalization of Alexa Fluor® 488 labeled LRRN1 E36 monoclonal antibody into the breast cancer cells at 37° C. (show up intracellularly as green), suggesting the potential for developing LRRN1 E36 monoclonal antibody as an antibody-drug conjugate.

Antibody-Dependent Cellular Cytotoxicity (ADCC)

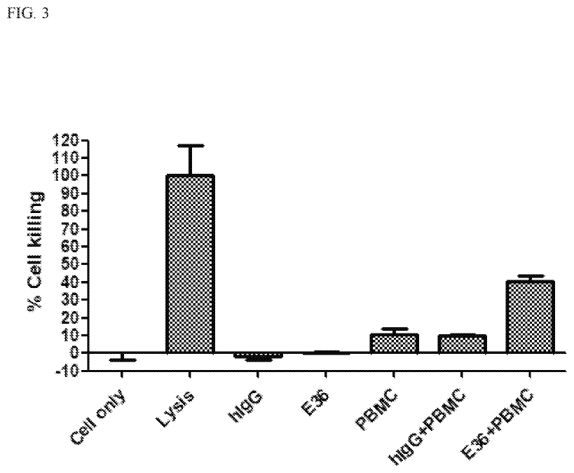

An in vitro study was performed using human embryonic kidney 293T cells with LRRN1 expression to examine the ADCC activity of the LRRN1 E36 monoclonal antibody of the present invention. The cells were labeled with DELFIA® EuTDA Cytotoxicity Reagents (PerkinElmer, USA) and cultured with or without peripheral blood mononuclear cells (PBMCs) in combination with human IgG (control) or LRRN1 E36 monoclonal antibody. FIG. 3 shows LRRN1 E36 monoclonal antibody of the present invention enhanced target cell killing compared to those treated with IgG alone or IgG with PBMC.

The result indicates the LRRN1 E36 monoclonal antibody is effective to inhibit LRRN1-expressing cancer cells by antibody-dependent cellular cytotoxicity

1. An antibody, or an antigen-binding portion thereof, comprising:
(a) a heavy chain variable domain having the amino acid sequence about 90% to 100% identity to the amino acid sequence of SEQ ID NO: 1; and
(b) a light chain variable domain having the amino acid sequence about 90 to 100% identity to the amino acid sequence of SEQ ID NO:2.

2. An antibody, or an antigen-binding portion thereof, of claim 1, wherein the heavy chain variable domain is SEQ ID NO:1 and the light chain variable domain is SEQ ID NO: 2.

3. An antibody, or an antigen-binding portion thereof, comprising:
a first heavy chain complementarity determining region (HCDR1) having the amino acid sequence of about 90% to 100% identity to SEQ ID NO: 3, SEQ ID NO: 9 or SEQ ID NO: 15,
a second heavy chain complementarity determining region (HCDR2) having the amino acid sequence of about 90% to 100% identity to SEQ ID NO: 4, SEQ ID NO: 10 or SEQ ID NO: 16,
a third heavy chain complementarity determining region (HCDR3) having the amino acid sequence of about 90% to 100% identity to SEQ ID NO: 5, SEQ ID NO: 11 or SEQ ID NO: 17,
a first light chain complementarity determining region (LCDR1) having the amino acid sequence of about 90% to 100% identity to SEQ ID NO: 6, SEQ ID NO: 12 or SEQ ID NO: 18,
a second light chain complementarity determining region (LCDR2) having the amino acid sequence of about 90% to 100% identity to SEQ ID NO: 7, SEQ ID NO: 13 or SEQ ID NO: 19, and
a third light chain complementarity determining region (LCDR3) having the amino acid sequence of about 90% to 100% identity to SEQ ID NO: 8, SEQ ID NO: 14 or SEQ ID NO: 20.

4. The antibody, or antigen-binding portion thereof of claim 3, wherein,
the HCDR1 is SEQ ID NO:3,
the HCDR2 is SEQ ID NO:4,
the HCDR3 is SEQ ID NO:5,
the LCDR1 is SEQ ID NO:6,
the LCDR2 is SEQ ID NO:7, and
the LCDR3 is SEQ ID NO:8.

5. The antibody, or antigen-binding portion thereof of claim 3, wherein, the HCDR1 is SEQ ID NO:9,
the HCDR2 is SEQ ID NO:10,
the HCDR3 is SEQ ID NO:11,
the LCDR1 is SEQ ID NO:12,
the LCDR2 is SEQ ID NO:13, and
the LCDR3 is SEQ ID NO:14.

6. The antibody, or antigen-binding portion thereof of claim 3, wherein,
the HCDR1 is SEQ ID NO:15,
the HCDR2 is SEQ ID NO:16,
the HCDR3 is SEQ ID NO:17,
the LCDR1 is SEQ ID NO:18,
the LCDR2 is SEQ ID NO:19, and
the LCDR3 is SEQ ID NO:20.

7. The antibody, or antigen-binding portion thereof of claim 1, wherein the antibody or the antigen-binding portion thereof, binds to a stem cell surface glycoprotein.

8. The antibody, or antigen-binding portion thereof of claim 7, wherein the stem cell surface glycoprotein is LRRN1.

9. A conjugate, comprising:
the antibody or the antigen-binding portion thereof, of claim 1;
operatively attached to a therapeutic agent or a diagnostic agent.

10. The conjugate of claim 9, wherein the therapeutic agent is a second antibody.

11. A method to inhibit cancer cells, comprising administering the antibody or the antigen-binding portion thereof, according to claim 1 to a subject in need thereof.

12. The method of claim 11, wherein the cancer cells express LRRN1.

13. The method of claim 11, wherein the cancer cells are glioma, lymphoma, lung cancer, pancreatic cancer, carcinoid, colorectal cancer, head and neck cancer, gastric cancer, renal cancer, urothelial cancer, testis cancer, cervical cancer, ovarian cancer, endometrial cancer, breast cancer, skin cancer or melanoma.


SEQUENCE LISTING (23 sequences)

SEQ ID NO: 1 - protein, 119 aa, Artificial Sequence
  VH of LRRN1 monoclonal antibody artifical sequence
QVQLQESGGGLVQPGGSLRLSCTSSGLSISDRWMSWVRQAPGKKLEWVADIEHDGRAKYYADSVRGRFTISRDNAQNSVYLQMSSLRAEDTAVYFCATWAGPNWRMDYWGQGTLVTVSS

SEQ ID NO: 2 - protein, 109 aa, Artificial Sequence
  VL of LRRN1 monoclonal antibody
ETTLTQSPGTLSLSPGERATLSCRASQSVTSSYLAWYQQKPGQAPRLLIYGASSRATGIPDRFSGSGSGTDFTLTISRLEPEDFAVYYCQQYGNSPLTFGGGTKVEIKR

SEQ ID NO: 3 - protein, 7 aa, Artificial Sequence
  HCDR1
GLSISDR

SEQ ID NO: 4 - protein, 6 aa, Artificial Sequence
  HCDR2
EHDGRA

SEQ ID NO: 5 - protein, 10 aa, Artificial Sequence
  HCDR3
WAGPNWRMDY

SEQ ID NO: 6 - protein, 12 aa, Artificial Sequence
  LCDR1
RASQSVTSSYLA

SEQ ID NO: 7 - protein, 7 aa, Artificial Sequence
  LCDR2
GASSRAT

SEQ ID NO: 8 - protein, 9 aa, Artificial Sequence
  LCDR3
QQYGNSPLT

SEQ ID NO: 9 - protein, 8 aa, Artificial Sequence
  HCDR1
GLSISDRW

SEQ ID NO: 10 - protein, 8 aa, Artificial Sequence
  HCDR2
IEHDGRAK

SEQ ID NO: 11 - protein, 12 aa, Artificial Sequence
  HCDR3
ATWAGPNWRMDY

SEQ ID NO: 12 - protein, 7 aa, Artificial Sequence
  LCDR1
QSVTSSY

SEQ ID NO: 13 - protein, 3 aa, Artificial Sequence
  LCDR2
GAS

SEQ ID NO: 14 - protein, 9 aa, Artificial Sequence
  LCDR3
QQYGNSPLT

SEQ ID NO: 15 - protein, 9 aa, Artificial Sequence
  HCDR1
LSISDRWMS

SEQ ID NO: 16 - protein, 14 aa, Artificial Sequence
  HCDR2
WVADIEHDGRAKYY

SEQ ID NO: 17 - protein, 11 aa, Artificial Sequence
  HCDR3
TWAGPNWRMDY

SEQ ID NO: 18 - protein, 9 aa, Artificial Sequence
  LCDR1
QSVTSSYLA

SEQ ID NO: 19 - protein, 11 aa, Artificial Sequence
  LCDR2
LLIYGASSRAT

SEQ ID NO: 20 - protein, 8 aa, Artificial Sequence
  QQYGNSPL
QQYGNSPL

SEQ ID NO: 21 - DNA, 50404 bp, Artificial Sequence
  human LRRN1
gagcacaaagcggggcgcaccgcgggcgccggcaacgagccggtgaacgaggcgaggcccgtgcgcccgcggctgcaagcgcccgcctggcggggagaggggccgacggcgtcagcccgggcggcggcatccctaggcgcctggggcgccttcctccggacctggccgctcgctgccccgccctctgcaccccacttctccgaccctccttcccagtcctgcctccccctgccctggcctctgagagccgactgagcccagccccgtgcagcagcggttgcctgtgtcgccgcctagtctccggtcttggtgctctcccgggggtgccccaaggagccagtgcgcgctgcgggctgggaaggaggcgccgctcagctagtcctcctcctcctcctcgtctttctcctcctcctgctgctgctgccgccgccgccgccgtgggtgccgggtccgcgcgcaccccaacacccccaccagctgggcctcggggtaagtccctggccggccgcgccgcggcaggaggtgtgggaggcagcgaggggaagtcgaggcaaacttggtggcgtgggagaccctacggggtctggggaacctcagggtaagatcgcgggcctgagggggacgagggggcgcggctcacggagctggggaaaatgacgaagtcgggaaacgagaatatagtgccccgtttccccccgtcccccttgccaaaaaaaaaaaaaaaaaggtgtgggagagcgatagttgggaaggcaggccagatagaaacaggcaaaaaaaggcagtgaccggagccgagcctgctggatttcaggggccgaccaggggagggaggtgcccggggacggggagggaggagggctgccgggatgtgaaccggggaaggcagctggggctggagagcagcgcggaaagggggcccagggagctggaaagagggccaagaggagggcaaggaaggtggcgggcgacggggagaggaaagaaaaagggtgtcttggcggtggccttggtaagagaaaggggcaaggggtataattgacaaggcactgaaagtattgaagtcagagccttgggaaggatctaccgaactctcggcggtccacgcggggacagacctcagcccgtgagccttgagctccacgcggggacagacctcagcccgtgagccttgagctccacgcggggacagacctcagcccgtgagccttgagctccacgcggggacagacctcagcccgtgagccttgagctccacgcggggacagacctcagcccgtgagccttgagctccacgcggggacagacctcagcccgtgagccttgagcccagaaggagtggcagcctcaggacgtttgccaggtggcctggaatgtgagggaagcctcagccccgccaggaacagagctggcgctgagttcccggctcggcccgggtcagaagcgaggaaagtttgccggcgagtgtcctttgtgctgcgtggagaggggtgtgggcggccgccgactcggcgcttccccgccgcccgcccagggagtccctgcctggcaagctggggctcggggaaaagtccagcagcgagagggccgcgtgtcccgggacggttccaggggagcccgcgcggaaacgcggaccaggcgcttcggcccggatgccggatccaggagaaggggacctcgcttgccgtgtccaccgccgcggccccagcgccaagaacagagcctggccgtgagtgaacgactggccggggtacaaatggttctgaagcacttgaagctcacaggcaaaggctaagagtctaaatcccatcccgccgtaagtattttcattcccaaacctaaatcgtaaaggaaaagtggaattctaattgggtgagggaacaggtgctgccgactctggcttttgcgtggaggattttctaaaagacaacatgttttgttttgtcttgccatgacaatctaaatcatgttagtggaataggctcattatttcatttttaaaagctagggctggaggagtggaaagttcaggaaggcactttgatcagtataaatctagagtaaaagtgttattggctccagggccatatgtgtcccgaagtctctgatttgcatgcaaatgtgagcggatcctggcagatgtctggcctcatccattcacatgaaggaatttgaaaagacaaggagtcttcagttaacaatagaccttaagaaaagagaataaagacgcagtgtgagtttttctgtttgggaattccatggtttgctgacacctttattcaggagccaaattctttaagtgctattgtattgaaagctcaaagtgctgcttgctctttacctggctgtatatattcgtgcaatattcaagctggatattctaagctagttaggaaaaggtttgtgtgaagcagacagtaactttaacccacttctggcggcttgaagtgatttctgaattgacccctcaacttttcgagttttcttttaataatttgactcttgctcaaatttggtcctcatcttcagagctattcattatagtagaagatcgttgtcgttgaccgacatgaagatttcctgagaatgtccactgtctagctgagaatgtccactgtctagccaaagtcatttcggttcttaaggcaccaaactgtagaactgcaggtgatgcaactgtcccaaccctagttacccgagtggttttcaggtcacataacagtgaacggggaggtagtcgtgcagtagactctggagcaagggcctttctgccgtgtctaacattagctgcgtgaccacactgcactgttcacctcttggcgttgtgcgacagctgaacaaaaggcaggcagtgtgggccgcacgtgaggcaggcgattatattaatacctacgactaaccagccccagtggcctcttcttttgtcaacctcaaacactgttgaagggaaaaagaacctattcacggaacagcatttgccccagtattctatatattcactgaaagggacattaaaatacaacacccccccaacccccgccagaaaaaaaagacaacattgttttggagggctcagtgtttcgggggaggggtggacaacagatgttagtctgctattttacatgttcctttaagttatttttgcaggttcagtgccttttatacataaggcaatattttcacaggatggggaaaaatgaattgaatcaaatttaagtaagaaaatgttatagccaagtttgcattttgtgtcttagagctatatagaagtcagaaagtaacaaatcattctaataaactcttaatttgactctacacgcctgatgtcttcctgatagagttgaagggtaacttaaaataaacttgacatttctagactataaaccattttgttgtaaagttatctagatatttttgaaacctggtaagttaaaaatgaccctcacatttgtgaaaccttatctttggcaattaacctgcctgtgtaatcgttattctgcaagtctaatgaaaggacagattagaactcattccttggtaaataaaactgataaagtgtttaattattagaaaaggatcattctgctaaataaaagatctcaagaccagatccaactgcagactgtgtttctaaaacataacctcagctagctctggttaatgtctgaaattctgggaaagcaaggaaaaactcaaccattgatctctctctggttagtctgtgctaaggccaccttcttgatctaaactgctagagccattaccacatattctaaagcagtgacactcatgggaatatactaggtactaaagaaaatcggtctctacggagttatattgctacttcccaagccagtggaatataaataataatgataaataatcagaatggtgggctttttataccaccataagtaggccataagtcataaataaaatatggtaaattataggatgcagatgccagttttacgagacttgcacaatgcttacttaaaatttatttttaagttacaaaataatagttccccgaatgattcatttcagcactaatgtcaggttattcaaatattgtatttaattttaaaaaattatgctgaacatctgttttgggttaagccaatctggagataaagcaaattccattgtagaatcatatgatcatttagaatcatatgtttctgggaacaatgaaagtatctttaactcatgaatagagctctgattaatatttaattcaggatgaaattatatggatgaagtcagaaaaacatttattctctcttgttttggctcaaagtacagaatagagataaaagttaaaccctatctgtaaaattcaatactttagtgaccaagggatccatttgttctaaaggtctttttaggtgaagtttgctttttaatttcttgttttagcatatattagcattcacctgctttacagatctgttacagaatgtagcctcccgagttctgaaacatagataaatgcatgataaatggtttattgacattgcattataatccatgataaaatgataaatgtttccagactttatgggcaccctgtagaactgttaagcatattatccaataattaaaatacataccattttatgttacagttttttaaaagaaacaatatgcaagctttactactatttatattttctactttttgaatgttttcaaagctctaggcaaatgtcgggagttattaacaattttatcagttgattggttggttagcaagctattatccagtcgtagctggatgatcttggatcagttgcttaaccccataccaaccttcaattttgtcatgtataacataatcgagttgggagaataacgctggaaggactaactgccataatcagtgagattactaaaaggtttatgtaagccatgcctccagtaccacaattaacatcaacatttaacttatatggaaccattgtacctaatgaactgaggatcattcctgagatgcggagaatctaggggtgaacttggaataatacattcccaaggtcctgtccactctgactttagagtaccctctaaagatgctgctgtatcagcttaagacacttagttttaaaatctatggagtgaaaactgcttttagagtggcttattgccttccatttatactctttattgctcagaggttagagtgttatttgaaagctccgattaatataaatgagttgtaattttccaacccaggctgtcctagctcacatctattataccactggcagacatgtggactccacagctgtcccctcatttgtgtatgaaacagacacactggtggcattttttggttaccatagttacattaacaatgtacatgtgacatccttctatgtgaaaggtttctgtggcagatattttgtagaactgggagtaaaggaaaactatttaaagtttggggaacacataaattagtctgccattgtattccggagccaccatttcaatccagggaagagagccatcctacagtggtgtccaggaaaggacaatcttggaaacaaagattttcaagggaggcatttccagcaatgcctaacctcttcaggagagtgtccatttaatattcatttcaaaatatttgctcttcctatttgtgactgccctttgtgagataatgtattagcatagtcattttcaatttgcagggggacagtgggcagcaccaatattagcacctatctcagtaagacctaggaaacaaaaaaggagtttgcactattaagcagtgagccacagaaagaatcatcctttagaaaaactaaaaaagctttaaaagagagaaaggtggcttaattggtttcatgcctataattttcccctctagaaagaaaaatgttagcatggcaataaagaatatcattaacctgcattgttatttcacaatagtgataagtcttagtttgcatccataataatcagtgtaatcaagtactgtacagagaatcaggaaggcgcaggctcaattcccagcaatgacactaatgactcaatgtctgaccttctggatgtcctgcaaccatcccggtgcttcatggctctatcagagttgtgcgttaaaggatccaggaacactggtagatgatttcaatatcaccagtgcgaataatttgatttctgccccaagagctagaattatatggagagctaatggtctgggtacagcccctacctttctgcaaggtgatgtttctggtccctaatcccatagctaagagattttgttgttctttttaaaattttctctccctagcattgacagtttctcaccacctcacctgggcccgcttgaggggaaaaattttaccaggggtacaggaagccatttacaatcattaaacatttgttcacctcacaagtatatattgagcacctgctgtatgccaggcattacagaaggcagtaaaactgaaataagttagcttgatttttctgcagcttgtacattggagcattttgtttccatcaacctatgcattctcctacaagtatttattgagtgctctgtaggaaaaaggcactctgcacagaggatttcaaccaagggtgatttttgcctctgaggggacattaggcaatatttggagccttctttttgtcatcgctgtcacaagtgagggtgggatgagggtgttataccggcatctaatgggatgctgctcaacatcctaaaatgcatagcacaggaagccccacgacggagaattatctggtccaaatgtcagtagtcgcaaggatcctgcctctgccctcagtgagcttcagtctagtcggggttacagaaaggagactgacaagtgcagcatagcgtgctatgatggtgggagtgcaggctacacaagtacagaagcagcagccttaactcagatgtcagctgccagcagaggctttaagaaacagactggttaattcacagaacaggctttcacctgccacacaagtgctcagcaggagacgattttccatctagcctctttgaacataagggggttggatggttgcttgagagagggtagactccttaagtttgtcaagaactttaaactgaagatggagttaattccttgtaggagttggtcagaattcagtgtgtcttgagtaaacgggagaggaggctggaaacagagagaaagtcgatagcaatagacataggacagcacaagctgcatgaagagaagatgcagctgagttgacaaagctgtcactgcctcatattttagagctttaaactttgccaaatccttaaatatttatctcatttgacctataacatcctgagaaaccaaatgtgattgttctttctccttttccccagtaactgagggacaagagtcatggagaggttcattgtgactgtggacaagtcactcgatgaagtttatctgtaaaaggagaggtcttgtgaggctcctcttcccagcacaagttgaatttgtctagggtcacccatctagggggagactggaggtgggataagacctgagactcctgtctctctcagacctagtgtcttccactcagatggtagcagcttcctagaccgtggagtcctaacaggtaacaagctgacggtgactcagcatgctagctcttgggttaccgaagaaagcaaggacctacgatgcatcggttaaaaagtatcatgcagagaaacttaagctgggtttcccctatacctaaggattaaccacgccccctacctaatggcttatcttaagctgaccttagccacttaaaaaaagacttgtgacaatctgggaagaaaggttcataggagagtgcagctagagttagaaaacgagatagaaaatgggacaacccagaacatgtgggaagaatggactgcctagaatagatgttatagtggtatcattttctgttttcattgatttttgttttttctttgataggagcgttttaactttcctatcagagaataatgaccagtgtatacagatatgcacacttacatgcacacacatcacacatatagagcaggggagacagatttcatacaagtttatgtttctttcttgtttgtttagtaagacttgcacgtcagaacgagttttggagagaaccttccctcaaggtcttatttctggtttctctgggataatgagtgtaaggtttcctatgagaagtaagactgagaagccacatgactactcaaggttcttttcattctctgtggtttccaggatcaggagtgagagttaggtaggacttcaaccatatcctgagagtttccaggacacatgtggaatctacatgaactcatcagaccatcagagaaccataaggacagtggagtagcaatgactggctttctccaggttgttattccagctgactgtggttctgtgcagtgaggactacatgtaaggcctaaccccccaaaggaccagttagctttgctgtgttccacagggacacagccagcctcctcacgtactcagaaactcatcctttctcatggggaggccatgtgaaaaatgtaggtcacttcaaagccatctgaaggcaaaacacaaaatatgtgggcaaaggctggtgggatatgtttctcttcatgcattcattcacactgtcaaattggagtcctggtgaacaagttcccaaatggcttcactaccaggattgagcactggagcggagattagttctatgcaaatgagatgctttagtgatcgagaggaaaatattacatagagctcccaaattctatccccagagcccaccatttttcctaacgtagcatacagtagtagcagtagaataatgatactctacactgtgcctttccaatgtgttgggacccatcaccaacaccttatgtgcattaactcctttaatccttgcaacaactctgtaatgtagacacccactttatagctcaggaaactaaggcagaccgcaactaagtaccttgcccaaggtcaaacaactagtgtgtatgtaataaatatttgtcaagtgaatgaatgaatgaatgaatgggtacacatctgttttagaggagagagtcctttatttgaattctcagtttatggcattcctgctttccaggcagagcattttcaagaactgcctctgtgacttgtgaatgataggggaagagctggtcggtggagaagcaggggttgaacttgggtctgtctgactctaaacctgtactgttaaagtgcttggctatatttcttcctctttaattgcagctataaaacaagaggactggactacgttagctctcttagcccattccactcaaaaagttacataattccatattaaagtctatgtaaaataaatcactttttttttattttactacttttaattttgaaatctagtttggtatttttatttaaccaatcattgaattatcttttaaaattatattagaattattaacagaacatatttataaaccctctttaaatcattttaaatggatgagaatgcatagttacagattgcattctcctaacccttaccatctcactgcctattaagttatatcatcattgtctaaatttaggtgtctaaaaattgaaaagcatcttttgttacacagggaaaagacaggtaaagtgattaattttgaaaaaattactgccgatatttgcagaaataagtaggttgctgaatctaatgaatagtattgaatgatttttttttctttaagatggagccttgtttctgtcgcccaggctggagtgcaggggcgtgatcttggctcactgcagcctctgcctccggggttcaagcaagtcttgtgcctcagcctcctgtgtagctgggatcacaggtgcctgccaccatgcccagctaatttttgtatttttagtagagacagggtttcaccatgttaaccaggctggtctcggactcctgacctcaagatatccacctgcctcggtctcccaaagtgctgggattacaggcgtgagccactgcccccggccatgaaaagtattgaatgatttttatgagcttcaaactgctagcaatattaaaagctactacttgagattcgcttctaaggattgttttgctttaaggtatcctcctgacatgcaagtgaagcatcagagcaatgtgtcagtcattgccgtagtgttacaggacatgcagcccaaggcaggggaaggagtgggtaacaaggcatgtcctagatttgggaagagttagtttcagaactgcttctcagctctgtgatctagagtgatttcatggaacctcagtatctacatctgcaaaatggggatgataatagagcccgcctcttagaatcactagaatcattcagtgagaacatgcatataaagtccttagcacagttcctcttccaaggtacatacttagtatacagtagcaatgagtagaagtggtatttgtgttcaaatctgacatttctgcctgctacaaagtctgcattctgaggatgctaattctgaggcaagtggagagattttaagggacaagaagactcagactagatattttatgaagtccgtagttgacatggttgtcacctttcgtggtggacccctagagtgctttatgtttgtctagacatgagaaatatggattccactgatgtgctttgaaagaattggcaaacaggtgaatacattagagccttttttgtttgtttccagattctcttctgtatcccactctctcttttctctctgaaaatattataatgcccctaaatggtgattcatggaatcacattttctaaataaagagaataggttaggttcagagatatcattccaccaagaggagctgaatttaagacattgtagcaaacaaacccaaaagcttcacctctctgcctccactgtgatatgaaggttggccatgtaaaactcttagaacaatgctgagcgtgttgtaaatggtccatagacaatgcagctaccattgttactgttaattcttagtatcatcgtcatcatacaatatcatcatcttgtatcttgaccaaaccacaaggcttctggttaggaccagattagaacagtgcagtttaaccacaaatctttctggaagcaagagaaagtcaccaggtatgctgggcatggtggtgcacacctgtagtcccagttacttgggaggctgaggtgggaggatcacctgaacccaggagattcaggctgcagtgattcctgatcacaccactgtactctagcctgagtgacaaagcaagatgttgtctctttaagaaaaggaaagaaagaaaagaaaaagagacagtcaccaggcaagccaaatcaggtgtgatccttagggagatgaacccacagaggccagttgctatgagttggcctccctggcacctcccgtgttttagctagctgattttcacttgcacatgaagggaagagttgagaataccattaacttgattttctttgaattctttgtgaaatggaaatagtaattatttgtggagaacttcattgtcttgtttaatcctcgtcatcagtgcttcccaattgctgtgaccacatttacaggttaattgtagatatccatactcttagctctgggggaaagccagtaccacacagctggtcacctggctacccttgcaggccaataaatagagttttctaagtgtgctgagaggaacacataccaactctgggagttatgtattatccccattttaaagatgtggaaaaaaaaccctagacaaggatcttgcccagtatcgtacagctagtaagaagtatgcataggaacaatactcttaactctggttttcctccagactgagttagtcacattgtccttgatcttcccacatctccttgtcctcactcaagtctagcatgtgttatctttatttacatcctgtagtaccaccaccaccaagcagcttcgtaagggaaaaggctatgactgagtcttaggtccctggcacctataaaagcatttggcacatggaaaatgtacttagcaaatattcagtcgttgaaagaggaaaggggcttccgtatcatatgtctatttcattttgccatattgctgctttaaaataatttgtacaaagttatttcatctgtattttggtagaaaatgagtagttcataaacctgtattatactcagagatattactatgttactgctttagagagcttattatagaaagttgaactcatttgggaatgctttttgtgatattattgccaaggaagtgttattaatcaaaaagtattcattaaatgcatgtcgaatatcaagtgctattagaaaggtgatatgagggttgaagaaaatgtacatggtgttatttctgtccagatgagcttccagactcattggggaggtagagtgaatagaatcaaatcaaaataattaagtgatcatctgtaggaattctgcaacaaagcagaaaaacaggggctcagaccagacgggtttgaatctcaactttgtcacgtaatagctgtgctcagtattggttaaaagactcaatttattgaccgcaaactcatctgtagaatggaggaataatgtctacccttagggctgctatgagaatgtatgggaaagcacttggtacaattcttagaacatgacagtcattcactgcaatcatttggcagatatctacagccccacttgtgtgcagatctttttctgggagtacagtggtgagcaaaacagatgatgtgtgttcctttcccatgtggagcttgctgtctgatgtgagataaaagcaataatccaattatcacacaagtaaatgtaaaatgatggctgtgttaagtccagtgaagaagaagtacacgaggccaggagaatgtaacataaagccttctaagagaagaccttcctaaagaactgacacttatttaagccaaaatctgaaggtcatgggagagttagccaggcaaagcgtatggagagggaagatctttgtaagccaaagcccagaggtgagaaggactttggtatgcttgatcaacagggagaagaccagtgtggcttagaatagaataaaagggtactttcttttctgcctacttccaaagtgcacttggagttcagagaaggcagaacttgtacaaagttcagcgaaggaagagactgcatcctataccgaagagatatgtcaccatgacagaaggcttgaattggtggagagggaaattgtgtgagtaattctattgagtcttggctggttaggtggcatgtcattgtcttacatgtgtggactggcatatgccaagtcgggcacaccaggaagaaagtttctcctccatttctgtgtggctttgagcatgctgattaacttctctgaatcttggttgtaaaaaaaaaaaaaaaaaaaaagatgtagcaccacaagtttgtgaagtttgtgggaatgggaagatataatgcatgtgaacgtggcacatagtgtctagtaagttagcttagcttattttattttctgctcctcataacctttttctccctgtactacactttcctttcattgtagtgaacctctcattatgatacttgctaactgagatcacatcacctgtgttgctcacagatacagattttttatagtagtttggtgatagccgaaacatttctcttgcttggcatgcaaatgctatcattctcgttcatgaactcattgtgtaaaatgcacttagccacatctggctgctgttcgcatgaacagaggtgactgctggtgagtggacaagatccttcacaagctttagatggtaatttcgtcggtgctctaatgaccataacacttagttgttattgatttgtcctcagtggagagatgtacttttctgcctgatcttaattatggttcataatattagatcatttcaacactgagttagcattttgcccttgtaacaatttgttttagttccatctttgtattgacttataagtaaatttagacactattaccaaatacttatcagaaaaagatagtgggggtagagtgggttatttttcagcaagatgctgagttatcctttattacatgctttatggcagtaaatcttggactcccttgcacccttttattactaacctgcttttctgtgtcccttgagatcttaagtcttctacttactagattatgagaaaaactcccaaatttccctccaaccataacctcacttccaaactccagtcctatatttccaaatgcctgctggatattttcaccctgaaaatcccaacatcaccgaaggagcaagaggaagaggtggtttccaggatgcagagtgaactgtggctgtacaagaggagagagaacgtccttttgcagaggaacgaagcccctgcctattgacccagcagggagggaaccaggaggagaagtactccagcctctcttttcccaccctcttctaatcattcaaatggaaactcaccccactcccaaaatgagctcctgccattgctggcccttatcttgttttgagaactgctatcatcccatcactcaggctcaaccttgaaattatttgtaacacctccctcccctgtctgcacatctagtcaactgcagtatccaatagatgcctctctagttatatttccctagtgtcttttaaatatggcacttcttctttgttcctgtcaacaccacccgaattctagccctcatcgcctctcctttagcttattggaagtgcttcttaaccagtttctccctatctagtgtgcctcctctccattctgacatagagctggtggattcatctttcggaattatagaactgatcccatagttcccctgtaaagaggcattaaatggtccccctggctatagtaatgtgctggatattcttgatgtccctccagttccacccagtacccctttccactttgctgtgtgccccaggaatcagatgtatatgggctgtacccataaatgggtcccttgccatctggcttcttgttgggttctgcatcctgcggtagctcactcagggttgtttgcccatcctttgtgagttgactttaacctcgcccatcccttatgctattgtttgaatgtatgcatccctctaaaatttgtatattggaacttaaaccccaaggtgatggtattaagaagtggggccttttgggaagtgatgaagtaatcagggctctgccctcatgaatggactaatgctcttataaaagagattgaagggagttccttagtctccttttgtccttccatctcctctgccatgtgaggatgtgaggacacagcctcagttttgtctttgcccccttctgccatgtgaagacacagaaagaaggcaccatcttcaaagcagaaagcagtcctcaccagacaccacatctcctggtatattgattttggacttcccagcctccagaactgtgagaaaataaatttatattctttataaattacccagtctcaggtattttaatatagtgacaggaatggagtaagaatcttaaaaatggtccctttttaaaacttttctgtattcattccctttttaatgtaccatctgttttcttctaagtctctgacaaggatgagaaatcagagaaaactccttagtgaagcataaaggccctccatatctaggaaccatcctaccttcctgtcttgatttccatagctactgtggcataaccaaaatcccattcaaaatgaaagcattcctattctgtcccctattccaagtctgctgccctgacgcctttacaccagtgtaaatgtttaacaactggctctccaaaaaggtaagccctcctttttgtgaccaattttaagctaccagtgtgatgtctctgaactcagatttggaaagaaatgctcacaactgactcagaataccactgctttgtgtacagctccccacctcccctgccactgccctcagtgccttacacagaagagttgctcaataaactattctaattcatttctatcattcctgtgggcatttggaatggtacagaaacctacttactagccacatgttgatggtaatatttgtgtacccagaattttttggtgagtgccaacataggtactagctggatttttgagacttgaaaacgctgaagtgaaaataaactaactacttgctcacattttgtgctaagttgctgaagcatagcatactacttaatgttgcttaattctccaataaaataattctcattttctgatcttgttttcaaaccatgtcttcattatcctcaattaaatgtaatttgaagtaataataaaccaccattaaaatgtatttcctcactattataaccatttgaatattctatagtttatgtccccttaaatcaaaaactctacagaaatttttaaggtacatgttcatcctagacatatatgattgcactgcaatttactctaaaatttattttattaagaaataaccctttcttagtctaagagataactttaattttgcttcatataaaaaatttctatattgaaagttcaaaatgcaactacaatttctattagaaaaataggaggtaagccaataaagatgatcttatcatagcattggattcttttgtattttaatggttatgaaggacaatatggatattcaactaaaattattctttttacttttaataatgaacatattagttgaagagctattatagaatgcactatgatttataatgcattttaatgttctcagctgttcctttgcaccatatgattacacaagcctagactgtgtaattaaggaccatccactaatgtgcactggtggatttatggtttccaggtcatcgatgatctgagcactttgagtattggaccattagtttcaaatgcatactaagataggggaacacattggcatcaaggaaaatctacaataagaaactctttcaaattcagggatgtaactttcacctgattttaatttaccaaaacaattaaaaaacctgattaacagaaatcctaattaactagatcccctccaccctcagtccacttttagaagaaaagggaagattggaagaaaataatttaaccagggctttcaattaagaaaaaaaaatattccaatatgagttcattattagggccttgaagaaaactatgtcttagtaaaaaatatataaccacccttattaaaggcaaaaatataggtattacaatttgaaactataaaagctttctaggaaaccttcagtcaaacagaaaattaaatttgcacattccctgagcctaatgctctagttaaccaaggtttcgctctacaagttagctttcttctacgtgtctaaaagaaggctgcaatgtactcttggggtccaataagacaattaaggcaggtggtatcacagaaaggccctgtgcaacgaacttgactttaacttcagctggttcacttatagctgcgggaattgtgacattacttgaattagtcatgatttgccagagaaagagaatgaataggatgggtgattgattgattgattgataagatagataagatacgatagagagatttattacaggaattagcctacgtgattatggaggccgagaagccccaccacctgccatctataagctggaaaactgatgttctgtcccagtgttgtaagaatgtaagtaaagcacctttcacagtttctgttttgtattctgctccccatccctgtctttggccacttccttaaagactcactgtggcccccaagctttcagaggaaaggtttcaacaagcaggcacacctctacttcattttcttgcggcgattaaaaatgtctagttctttaagtccttattagaaatagtcataaaaaatagtttagaaaaggaatttgaaatcaagtttattttaagagttcaggggcagatggcgttactttgcttctttaaaaaaagatggcattcaatgtggttgagttctatcaagtcaaaggacaagtactgtctcattcacccaaacactgctgtctttaaccttgactagcgggaacttctatgttaaatgtatgcacacttctgtacatctgccttcaaattttaaaggaacattttaatgtggaagggatatgttcttgacattttcttagtctaaagtttaaacagcacttcgtcttctaattaggaagccctaagcagtcaggctctttagagaaatatcactagatcagagctaaaggctcccgtcttaaggacattggatgttagttttttttttttttaaataaaaatgttgttaaaatgtcagataattagtacagattactactggtttaaaaaatataacaaaagacagcgcagagctctagatgagcagagaaaaatgcaataaatgttctgaaatgaatgtgaaaatgttagccattttcttgatgaggagaattatcctcaccattacaaactaaacttaattcagtctgggagaaaagcaaatctgatgtagtctaatggaattgcttgacttgatcagcatccaaaaatctatggctcagtattagccgcctacatgttggcactttcagtgcccacagtgacgagtctattttttcactggtgtatgtgtagtgttgaatgtacaatccggaaccattgtagcaaccagttccttatgcaacacttactaacagggcatggcgaaatctgtccgaagtgagaatacccagtgctatactgtgaaagacttaaacagaagtaatgcaactgggccttccaggcatattaagcctgccaaagtaaagatttggcatttaacatttaatcatgcagctaattacaaagcaattatttcatatagacatattacagggagagaattaaaataatctgtaaaagactaaaattaattgatttcatggtagtttttaataactacacttgctggtgctgaaaaggagcaataaaaacagcccagttgttggttgatgttttgttctgtgctgggccttctgtcagtttctcaattcaataatgttaaattgccactttcatgattccagctatgcaaatagtctatttcttaaaaagaaaaaaacaactttcttgatcttacaagtgatttaaacagaaaaaaatttttcccgtcgtcctactttctttgcctgtcatagaatcttgggtttaaaagggctgtgaaagactaaaggctcgagattcttcaaactgtatctggtgtgtgactttcaaaacatgggaggaaaatgattttggcacagagctaaggtatttcttgaataattataagtttagcctgctgggtcctagaagaagttataaagatcatagagcttaccacagcttttaaagttaaggaacaggtacctctgtctccctatctgtaggatatacaggcaacagtagaaagcaaactcttttcataatcagttgtgtgtagtaagagtacaatggggagaagtagggtaaggaaagtcaggactctaagtatttgataaaataagcattcatataaaaataatttagatggcagttaatacacatattaattaactactttacttttattttttttgagacaagagtctcactctttcacccagactagaaggcagtggggtgatcacagctcacttgcagccttgaactcctggcagtcctcctgcttcagcctcctaagtaggtaggactataggcacacaccgccatgcctggctaatttttgtattttttgtagggatgggatctcgctatgttagcaggctggtcttgaactcctgggctcaagcaagcctcctgcctcagcttcccaaagcactggaattacaggtgtgagccaatgtgctcaacccaatattaactactttaaaatcttacacttctttagtttactagggctgccgtggcaatgtaccacaaactaggtggcttaaaacaacagaaatttattatttcacagttctgtaggctagaagtccaagatgaaagcatcaaggtgtcaacagggccatgctccctctgaaacctgtaagagaataagtccttgcctcttagcttctgggggtttgctggcaaactttggcattcctttgtttacagctgtggaactccagtctctgccttcattgtcacatggtgtttttgctgtgtgtccctgtcttcacatcattttccctcgtctttaaaggacaccagtcatattggattaggggcctagcctatagcctactccagtagtctactcctcatctaaacgaattgtatctgcaatgaccctgtttccaaaaaagggccacattctgaggtccttgggattgggacttcaacatctctttttttgaatccacaatccgtaactaacaagcattttgacccagatcatgggatacactgatggtaaatggtgtaggcattttgtgcatattaactcatttcatctttttgacaaccacatgaggaaaggatgctgacactactctcaaaggaagacacagagactagaaaagtatcatgtgtggagctggattttaatctcaaatctgttgactccgaagcctatgctcattctcgtattctaaagagctgccttcccagggatcctgttttcccattctcttttgttgttgttgttgttgttggaaaatctagagataatttcctgagttctcaaaaaccatgtctacctttctcacaataatattaatttgccctgctagtttttagaattacttttacttcatatgttctgtaattacattgtccaagaatgtgatctagaaatgacatatatttttaataggaagtaactaaaatgcctaaagtacaaaacacctaaaaggcatatttgtgcattcaacagtcattttcgagtacctgctctagggctagcattctactaggccctgtctagcaaatttgccatcaaaagctcttaatttctaaaaagtttcattcagccttttctttagggaatatggtgggagtcatggcctttcatacagggctgaacattttttgtttaagttattttctatgtaaaccgctcttcatttattcaaatacaataaataaggagagcaatggtgtactctgatacttgaaaagaggagatctattctgatggaaacctttgcttgcttccatcctaagggtttaggtaaaagaattcttatgtatttccttctggattcaaattacatgaactttacgagtccatacacctgtttcagacaggctttgttctcgcaggtatagcgagagaaatgcaataaatgaagaaaggtctaaatatactttgtgatgtactctggttgttgtttcattgaacacttgcaggctagtcactgtgttaagtgcagtacctaccaggatgaataaaacataaccttggacttcaagagtttgagtcacctctgagaacactctgatgagtgactggaagaatgaaacagtgtggcttagagcaaagccatggctacctctgtctttgcagttaccaggccccagctgacatgcctgctggatcctctgagcctgtgagtggtggcaggtcagagccagactaaggctggaatctttttaaacgtggtctgcatgacaggactctggtgctttaaaaaatatacagatattcccagaccccatcccagacttactgaatcaaaatctccatgaggcatgagaatgtgcattcccttaaaacgatctccagattcatgaacaactaagtttggagatatagggctgtgcatgcctagtgtggtggccactggttacatctggctatttaaatgtaattccagtcaattgaaataaaatgtgaaacctccatttctcattcccaccagctacgtttcacttgctcagtagccactcaggagctaccatgctaggcagtgcagactgaatatttctgaagttgcattagatagtgctaatagagcaaacacaaattagtaactctcattcctccctcccaaagatccacctatctctttactcctcccttttgacatgtaccttttctttttttttgagtaacttgtgtacttgtcttatacactccccttaagattagaagctgtttaagggcagggtctggtcttcctggccctgtgacacagcaaacactgaatgtatttttaattaaatcagtgtgattcaatttgcaccattttattggatctcttttgcctttcaactgacactagatttcatcagagtctagagaaaaggatggatcacagaacaggtttgtctcaatcacaaacagtaggatggatttttgctaaattgcaaggttttcttaagcacaaaatgtagaaaagacccaccagagctgtgagtatttgcaagcctaaacctattgtcatatttgggtacctagaaagcacatttgaattgattcagctagctgtttgatttatctattgagtgcccactatgtagatgccagactcattgcctcaccctggaatgactcagagtgaacaagacagaaacgggttcctcactccatgagatgcacactctcatatggcatattgaaatattggcaaggagtctccctgcccagacggattgggatagaaaagcgatgcgatttgggttctggcatcagatgcctggattcacatagaacctcaaccactctgctgtactagttccaagaccttgggtagttcattttatttttctgagtctccattccatcacccataaaaataggaacatgatggcattcacctcatagcgttgcaagcattccatgaaataatttatgtaaatgattcaatgcagtgcatgttgcataataagagcttaataagaactaactgctgttactcttacaactactactcagccaaaactaccctgcaattaacattaagatgtaaaaaattaggtgtctgcaaggttagaaaataactcattatttggaaagggtgttttctgtctttcttgagctagcagtttaattcctgtagcttactatttacatccttgcatcctaaatcactgagatggatatataaatgttttggtagatgcctatctcttgttttattttttatgtttctttggtgaaggatttaacttgtttgaaacacttctttccagacaattaagaatagggaaatgcttcaggtgtcattcaacctatcttggtgtagagagatcacaaagaattctttaaagaggcttaccattaaactgcaaaactgaaatcctaataactagaatgcaaatatttaattagcatttctcattaaaataaatgtgtgactttttttcttaccttcaaaaatgacatggcctttgaaaatacatactggaaatgagctgggaagagaagggttggattttgaaggcagccacttgatactggggagtttgcaagttgagccttcctgagtccttcccattctttcatcggggggagaagaagactttgctgtgcacctgtatataatagtgaaattaatttcaaatccaggacctgctttattcttacctcagattccatgaaacctgctttaaactttttctaatattctagaaatctactctatccttacccaagggatagaggatatttctgtggcacccaagctattcagtgaacagatgaactgtcccagggtcttcactgtgattggaggggctcaagtcatttgggaaaaatcggtgttggcttcaagagctataaatgctcccaggtagccagactatactattgatacttcattcatttgattcaaaattgggggtttttgggatgactgtgtgtcctgtgggaggtggtacttagagcatgggctttgaaaccaagacgatttcatttgattccttccttcatcactttctagatgtgtaccttgagcaggtatatctctatgcatctatttcctcagtgtaaaaaggggacaatcatagtacctagctcctggggatgttgtttaaaaaaaaaaagaagaagaagaaggttaggttaaaagaagaaaagttccagataattttcaggagattgttttcaggacataaattaattttcttatcggaagagtagatattaacttaggttttcagcacctgactctaattgtcttaagttgacgccaaaggaaatttgttgaaatcctgctatgatccactgctcaagacatgttatctcatttaatcttcagaacatccaaatgaggtaggtataactatgcccatcaaaaagagggggaaaccagggcttagagaggtggagtcatttgtcccaggtggcataggtagtaccaggatatggacccagctctgtctgcctcttgagactgtgcctttctttttaatttttttttaaagctaaacacagtttctcttaaaaatctgggagcaagtgttttgctcccaatcaagaagtgctgcttaatgggtacctttaagagcagaaacctgaacaaagcatcaaatactcaatacctggagctggagccctagtttcagcccagcctcatcatttagaggtaggaaagatggctaacatgcactgagctggagtcaagaccagaggtaaggactcaggtcccctaactcttggtccagtgctccagcccctgtaccgtgctgcttctcaaactacctgagagtcagataggactcagaaccagacagcattcaggcatgggccctatgtccatgacttactagctcacttgagacaaatgagttgacatcattgtaaagaatagagatgatcataccatatacacgtaagtcactcagtgtagtggttagtacatagtcttttattaaagtgagtttactagtagagttagttacattcatattattcatgagggctttcctatcttgaatgtccaactccaactcctaattattttaaaaatctatgcacttctttcacttgaatttctatgccctcatagactttacaggcacatgttctatcaccaattaagagaaatttaacttacacattttacaacttatacatgcaatttggctgtgcttatacagcacagatcaacattacaatcaatttactgccatgttaaaaaaaaaatcagtgaacattgttcggctgacctgactaattttttgaaataagttggccatcagtgtaattgattgagctatctgtacaacccccaacctacctaagcaaaggtcaaaatggaggttattaaagaaaaccagccactgaactatccctaaattgagtgatgttttcctgtgctactgataaatgattcattgatggagcactaaagactaatgtcattcaaagaactgtagaccctgccctcgtggcaaagaatatgaatttaaccttagacaaagcaaagcaatgtgggattgtctatctagagtgcaggacagggtgcttggagagtggagcttggcctccaaatatgtgacctcacctctctgaggtgtcctctgcaaaatgagaggttattgtagatgtgtcctgtgatctgtaaggccgtctctacctttcacagtttgcgcatctgtatatttcacagggaggctagcctgcttgcttttgatcagttgaagtcaaatcagactctgaggtcgacgttattccctagggctctttcatagacacccttcccgtctgtcccaagacaccttcctgggtttggattcatgccagctggcactgggcttttggcttttcatttttcttttgctttttacattttctgttgtaattcttaagtccatccagtccagcctaccctgcttcccatgcctggccaaccctcctgaaagggctaaagtgccatccatgcccagttcctcttaggagctgaatgaattgtgaccccagcttttatgctcgacttggagaccagctgcgcctttgggcttttcccagagatgttggatgagactgcacctgcagtgtgatggatttgcccttgcctgaaagaggaaggaaaatgagggagaatcaaaagatcacttgtatccatccccttctttgtacactagtacatttatatcaccagagaatttttgtctcgatgaattaatagtaataacatacaagaaaaaggagaaaaggaaaagaaaattagggggaggacaatctaattttctctgggagccagtaaaaccaccccagaaatttaacatggtcagattaggaaacactgaatttgggagcaaggcactcgagtttcattttgttcccctgacaattaactgggtcaatttggatcacctaggggatctctaaggtcctgactaccttttaaagtaggtcttctgaaagaaaggagtgtaaaatgaatttttggaaatgtaatgcttcttaaagcactaaagaagttttcttctgagcagagagtcacaccataataaactgagtttataattatagattttcacgtatggagtatttgccaataatatttgaaaatttatatctaatcatctagttatcatatattccctttagctcaccattatagatgctatttttatacttatattcagaggttgcaaactggccatgggccataaagggactctgaaagcaggtaggttctgtttggcctgcacagctaaaacattttgggttgccttttggaattcatgcactctccagccttctacatgactactgcttcttactgtgttagaatcaactccctacacctacctggccctagaaagcattaagtttgagatctttgtcacaatcaaactccatccctccaaaacaatttaataacacaagaggaagaaaaaagaagggaagaaaggaagagaaagggagggaggaagcattgcctctctgaaaatgattggaagtaaagttttccatgacaccagaggaaagggctgatgacttcctgaggttcctttaggcctgggaagctatgactacttaaggtaagaaaaataaaataggatacaaagttacatgacttgttcaaggtcgtccagaaagtttatgctgaagccaaaattaagaattcctttcttggcgaacatccttgttctgttgcctggcctattgagatgtgtggccgccctgcttggtgatgaataatgtgggcttttaggaacagttccttatgcaaaagagcctcaggaagatccattgtgtccttgcaaggggatacactatattgtttccttaatgcattacggagactattatagctcttcctctcatacgaccctccatctgtgatcacttaacacacactacagagtgttggctttggataatgtgtgaaccaggaacataattttggaaaatggttggagaaagaaatctaagagtcagaaagggtcaggtgccagaaaaaagtctaagaaactaatgtttttaaaaagagtctgtgcagattcagcgttcagatcgtatgtgcaaggccctctttccttcccaagaggaataaaatacatcctcaatacaaaatttactgtaatttctctctttgacccccctgtactggctagctacattatttaaggtacagaacttgtactggatcataccacaaaaggcattctctactgatttcaacgatctatgccttactttcatcttcaacacccatgaaaaagactcagattcaatacgagacatcaatcctgtaaataatagtagctaacatctactcggggcttgccatgtgccaggccctctgccaaaggctttacatttaacagttcattctaattctctcacagtccttataaagcaggttttataatctccattttcttaaagaggaaactgagacttagaaagtttcaataacttacccaaggtcacagagctgaatttgaaaccaagtctgtctctccagaagccgcattcttaactttatgctatactcttgtttcagacatctgaaagcccttgttaagtatcacaatcagcactggggcctgtagctgaaggcagagtggccttcagggacaggaggatccttctgggaggtcagtccacacagtggagtctgggtccatcttgcctcatggagtagctacaggcaatgttggtgttgaggcaatctatttttcttaaactctgtaaggcaagaaggaaatcttggttgaacaaaaatcccatttccccattattcaagccattcctaaaagaaaggaagcaaatttgaatcctgcatttgccatggatctatttggagcctgagaaaaagctaaaagaatgatgttttttgaacattatctacatgatatggatttggccaatccagaaaggatacataaagctcatgccctcagaaggcatttttcttcatttggggatcaaaattttagttctttgctgtgattatactatctatgagatggtgataatttccaggttggaggtttgtgttttataaattgcctggatagggtttcaatttagctttaatacaatatttggtctctggattttattaatttcaatgataatggttgaaaatataatagaaatgacaaataatattgataataactctttaccaagtatctgccatgcccaagggccttaaagagatcattcattactcccaataacaagatgaaaatgaaattcaaaggcaaatctgatagggttccagaggttcaaaaataagtatttgagggctcactatgtgctggatattttcccagtgtgttagggttctcttagagggacagaattatatatatatatatatatattatatatatatctcccattagtgtatgtgactattatatatataaaggggagtttattaaaccaatcacaaggtcccacaataggctgtctgcaagctgaagagcaaggagaccagtctgagtcccaagactgaagaacctggggtctgatgttccagggcaggaagcatccagcacgggagaaagatgtaggctaggaggctagacccgtctctctttttcacgtttttctgcctgcctttttttttgctggaaggtgattagatcgtgcccaccagattaagggtgggtctgccttcccactgactcaaatggtaatctcttttggcaacacccacacacacacacctaagattaatactttgtatccctcaatccaatcaagttgacagtattaactatcacaagtccatcccttgttaacttgaacacacatctcctgagatcatacattaccttaaatagagacaataatgaggtcataattacacctaacataactatccttcccacaaccggaaacacactaatccccaacccaaatactgttacataaagttaacaatacttaaatgctgatatgaagtcaataaatcttatgtcacatgataaaggaaaaaaatgaagatattttcttagtacaggtatatacaggcacaaacgtgtttttaacaaggaggaaatactcatgacagttaacagtccttgtttctgcacttgtttctgcagctggtcatgtgatcgtagctggtattgatgactaccttcttgtactacccattctatattccctttgccttcagcaagcacctcagcaggttgtgttttcttttctttctttttttttttttttcctggtggagtgacccaaaccttcattcctgaaaggtctgggccatttgtagtcctttctggattgggctgttgtagttttccattgaccttaatcacaggacattgtaatactaagagatatcctaatggatctcctgtattccatgcatactctcccttacttccatcataaagtagtagactgattccattttgataatctgggtcaatcacaccagccaaaactgtaactcccttcttagcctgttgacttaaaggtagagggagcccaaagtgtccaggtggcaatcttccagtttcatggaatcgttgttatgtctcctggtggcagcgttcctcctcctggaactaagatgtctagggtagcagaatgtaatgtcgtgggaataggaaacaaaaattttgctagtggatcactaggggtgatggtgagtggcgccacttccacctgttgattcctggacccatgaattctagctatgggacaaacattaccatgtattggatgctgattcagagcatacacagccttctggagaactttgctgcagccctgcaaatattgtcacctagttggcattttaattgtgacttcaaaaggccattccactgttctgtcaatccagctgcttcaggatgatggggaccatggtaagaccagtgaattccatgagcatgagcccactgccacacttctttagccgtaaagtgagtgtcttggtcagaggcaatgctgtatggaataccatgatggtggataaggctttccgtgagtccacagatgttagtcttggtaaaagcactgcatgcaggataggcaaactcatatctggagtaagtgtctattccagtgaggacaaacctctgcccttttcatgatggaagaggtccaatataatcaggtagctggctgatcatcccgaggaatggtgctttatcgagggctcagtgttggtctctgctgctggcaaattgggcactcagcagtgtctggagccaggtcagccttggtgagcagaagtccatgttgctgagcccatgtttaacctatatccctgccaccatggccactttgttcatggactctttgagcaacgacaggggtggctggggaaagaggctgagtggtgtccacggaatgggtcatcctatccagttgattattaaaagcctcctctgctgaggtcacccattggtgagcactcacatgggatacaaacatcttcacagtttttgaccactcaggtcaatccacatacctcctccccaaatttctttgtcaccaattttccaatcatgtttcttccaagtccctgaccatccagccaaaccattggctacagcccatgaatcggtatatactcacacatctggccatttctccttccatccaaagtgcacaaccaggtgcactgcttgaagctctgcccactgggaagattaccctccaccgctgtcctttagggatgtcctaaaaaggggctgtagtgctgcagctgtccattttcaggtggtgtctgcatattgtgcagaaccatctgtgaaccaggccctagtcttctcttcctctgtcaactgatcatagggaactccccatgaggccatcagggcaggctgggggagagaaggcagggtggcctgagtggagaccatgggcatttgagccacttccttatgtaacttacttgtgccttcaggacctgcttgagcccgatcatgtatataccacttccatttgatgatggaatgctgctgtgcatgacccactttatggctagatgggtcagaaagcacccagtttatgatagttcaggttgcatggtagcttggtgacccatggtcaaacgttcagtttccaccaaagcctagtaacaggccaagagctgtctctcaaaaggagagtagttatctgcagaagatggcagggccttgctccaaaatcctagaggcctccactgtgattcatctatgggggcctgccaaaggctccaaacagcatccctgtctgccactgacacctcaagcaccattgggtctcctgggtcatatggcccaagtggcagagcactttgcacagcagccagggcttgttgtagagcctgcttctgttctggaccccactcaaaactggcagcctctcagttcacttgataaatgggccggagtaacacaccccaacgaggaatgtgttgcctccaagatccaaataggcccactaggcattctgcctctttcttggttgtaggaggggccaaatacagcaacttatccttcaccttaggagtatctcaccaggccccacaccactggacccctaaaaattttactgagggggaagattcctgaattttagtcagatttatttctcatcctctggcacgcaaatgtctcaccaataagtccattgtgtttgctacttcttgctcagtggatccaatcagcatactgtcatcaacgtaatggaccattgtgatattttgcggaagcaaaaatgattaaggtctctctgaataagactatgacacggccgggcgcggtggctcacgcctgtaatcccagcactttgggaggccgaggcgggtggatcatgaggtcaggagatcgagaccatcctggctaacaaggtgaaaccccgtctctactaaaaatacaaaaaattagccgggcgcggtggcgggcgcctgtagtcccagctactcgggaggctgaggcaggagaatggcgtgaacccgggaagcggagcttgcagtgagccgagattgcgccactgcagtccgcagtccgacctgggcgacagagcgagactccgtctcaaaaaaaaaaaaaaaaaaaaaaaaaaaaaagactatgacacaaagctggagagttgatatacccctggggtaggacagtaaaggtatattgctgtccttgccagctgatggcaaattgcttctgatgggccttatggacagaaatggagaaaaaggcatttgccatgtcaatggctgcataccaggtaccaggagacgtgttaatttgctcaagcaatgaaaccacatctgctgcaattggagtcacgacttagttaagctcatgataatctgctgtcattctccaagatgcatctgtcttctgcacaggccaaatgggagagttgaatggggttgtggtaggaatcactacccctccgtctttcaagtccttggtggtggcactaatctctgcaattgctccaggatgcaatattgtttttgatttactatttttctaggtagaggcagctctaatgccttccatttggcctttctcaccataatagccttccagtcagggagaaaatgtgggggttctgccagctgccaagtatgtctatgccaattatgcattctggcattggggaaatgaccacagcatgagtcaggggacccactgtaagttggacctgagctaaaactccatgaattacctgacctgcataagcccctgctttaactggaggaccacaatgatgttttgggtctcctgggatcaacatcagctcagagccagagtccagtagtccccaaaatgtctgatcatttccctttccccagtgcagttaccctggtaaaatgctggaggtctcctaggggaaggatggaagaaagattcactgcataaattgttggtaatgtagtggggtttttccttaaagggacccagcctccccttcattcaagtggttctggttccataaactggctccagtctggaaattgagcggtcatgattctctgtttttataattcaaattagtattttgtccatgtgacctagaagttttctgtttgtgtaatttaagtaggaatgcagtaggcttcctatcaatttcacttctaggaacaccgtgattaattagccaatggcagagctctacatgagtcagattattctgattgctgctttgcctctgctgtccattacagtagctatgcccactttgcctttgacagttgagtgctgccacttggcccctgccaccttgggatccaattcttcccactgtatttaaattttgtagttgagtgactgctgttcccaccgttagatctgacatacagagaagagcaattacagggctcttcagagatgcaggtgctgccctcacaaatctattttgcagggcattggtcaaggctatatcttctgggccctcccaactgggatgagtgggtctaaagtgactaatccactccaccatctcaatctccctaggcctttggatcccttcctctacattaaaccaagggagatcaggcatttccagcttgctcactgtgggccatcttttaatccatacttcagctaaccaagcaaataaactattagaatcttttcaacttcctgagctgcaacattaaatgcagagttcctacttagtcggcccaaatcaataagttcagcctgatccagctctgtattccttccaccattatcccatacccttaatatccattcccaaacctgttctccagattgctctttatataattagagaattcaaacagttcttttctagtgtagtgcagctcctcgtgggtcacactctcaacctcacctccaggggcccatcgggattttagttataggtctagaagcaaacaggggtattaggggtggctccggaggagaatcaacattattttgcctggcaactgcctcaggagagaggccatcactgttgcctcaggcagcgcagggtttgtctcctcagacaaaggcggaaaggcctatggcagcatgtgttggggaggggatgttgccactactggggatggggaagctgtttcttctggcaaaaaaggtgcatcagagtttacaaacgtagtgtcctcagcttcatcagggtgctcccacacatccccattccaagttgcagggttgcattcttttccaatcagtgccctcactttaacagtagacacttggtgaggctgtgcatgcacctttcattgcaggtcacccactcgcatgataagagtttgtgtctgtttttccacagtttcagctctttctgtacagaagacaagactctcactcagggtaatcttagcagatttgaggctcggtatctgcttctgaagccaggagacagaatccctgagtccatcattttccatcattttctttcatcactttgtccactgaacttaggagcaaccaaccagcttcattgtgttcctcggttctccacatatggtcaaaggcattatgtatagagtcactgaactccttgcctcttacaagcagtgaatcaggagtgtcaaatgcatttattttgcataagtctctaaatagtttacaacaacgactatcagtgttctccatactattagaagtacagtccttagcatttttgggtctaatcatattaagcagccaactccagaaaccccaaaaccaatgaaagaactccatcctttaatattctgttcctttagaaccactcctggtaccaaaatctgtattagcgttctcttagagggacagaacaggatatatatatatatatatatatatatatgatatatatattattatacatattatatatcatattttatatacattatatatataatatatgtaaaggagagtttattaagtattaacatgcatgatcacaaggtcccacaataggctatctgccagctgaggagcaaggagaaccagtctgagtcctaaaactgaagaacttggagtctgatgttcgagggcaggaagcatccagcacaggagaaagatgtaggctgggaggctaggcccatctcttcttttcatgtttttctgcctgctttatattcactagaagctgattagattgtccccaccagattaagggtggatctgccttccccagcccactgactcaaatgttaatttcttttggcaacaccctcacagacacaaccggggttaatgctttgtatccctcaatccagtcaagttgacactgggtattaaccatcatacccaggccctaggctgaatatttttacataagtacttttttgttatcataatttacatgtatgaaccatatatacttagccttaagtttagctatggaaactgaggcacagagaatttaagtacagatgcttctcagcttatgatagggttacgtcctgatacacccatcttaagttgaaaatatcattatgtttaaaatgcatttaatacacctaacctaccaaacatcatagcctaccttgaatattgtcagaacatttacattagttttattgggcaaaatcatctaccataaagtctgtttgctaataaagtgttgaatttctcatgtaattgaacactgcagtgacagtgaaaaataggatgctatcatataggtactcacagtatggtttctactaagtgtgtactgctttcacacctttgtaaagtcaaaaaattgtaagttgaactatcgtcagttggggaaggtctacagcttgcacaaagtgacacatctagtaagtggagaagcataattcgaatacagaacacttgattcctccagggtagaattctttctccaaccccagagctgcctgatttttttttttttttttttgcagtaacagaaatacaaataactagcttgttactgtgagtatatatttctttttgcccaacttcataagaagttaatgcatataaatcatagagctaagtcccaccgaataatgtcattctgttaaactaatatcctatctgacacaatgctatggggaaagtgagagtctccgccaagtgcagtgatggtgatgatcatgcagttcatttccttcagaattttatactcagaacagagtgtcccaaacatatcgtgttggaaaaaaaaaaagaagcatcaaatgtaatgtataattgtttgtgggactcttgaaatgcatcccgtgccaattctgggccttaaagccatgttttgttaattaaaggattgtttctttaaactgccagacttctttgtggaacatcaccctggtaattagcaggacattttcttcttacatttgatagatctagtcagtcagccctagaagaaagcaatttcacttaaaaaaaaaaatctattccagccatttcttatttgttgcagaatttaagataattaatttgtatatgttataaaagttggcctgaattattatggcttcgctttttttgtggtaaaaacacttaacattaaatttaccatctctcaattttaagtgtacagttcagttgtgttaagtatattcacattattgtgtaacagatctctagaactttgtcatcttgcaaaactaaaactttattcctattaaacactaattccctctacctccaagcccttggcaaccacctttctactttctgtttctatgattttggctactttatatgaatggaatcacgtactattcacgctattgtgaatggcttatttcataatgtcctcaagattcatccgtgttgtagcatatgacaggacttcctttttaaagctggatagtattccattgtatgtacacaccacattttcgtgtgtgtgtgtttgtgtgtgtgtatgtagtagtggtttttactgattggcacaagatcacggcctgtgcttccaatacccttccctcctatgcagctcaaatggaaggaaactgattaacacataggaagcaatagcatgcttacaggtttaaactcctttcgtttttacaaaaaggagtgggatatggggggtggtacaattttaggggtagtagcaaaactctccctagactacagtgggtgggcctgtctgtcccctgttgctggtaccacataagaaggttcatcagtaaaggaaaaaagggttacatgtggttggcctgctattaagaggggaagataggatggtatctgctgtgtacctgtgttgggccaaacctgggactggaccagtgccccttttttcttggcccacctacacaacccagctgtactgtctcctgcacccatgggaaggaggtgtgaaggaagctgagaacatccgccttgagaagatgaaacaggaaacctcactttcctttcactttctctggactaaattccactcccctcagctctggaaagcactgcaaaagtcttccttaggagtgctgtgaagacgctggggaagatgccggcccaggcaggtggccctgagtgtgcaaaaagaaaaacttgactttccatgcagtatcaagcaaggttactttgtaaacaaacaaaactggaaaataagactacatactaagtccttgagagcatttggattgggggcaagggctttattactccttcccccccccattttataaccccacaccaagtgtcaagagctttaaggaccacaaccaaccccaccttcccttcaagcaccattccttgggttcctcagttttgttgagctcatttggggttattcctgccaccctcttaaaaaatgactcagcctgctggttggtccattttctaccctttctacaattatgaaacacactctctaaataggagatacataagccctcactctatagtccaaggaactaggtgatataatgccaggaaggctaaattgtttgctttcctgggtaatcctgatgtgaatggacagcagcagggttctggtgggatcagagcagtctagtttgagccaccgcaccacatttttttttctttttttttaattaattttaatttttttttttaaagctccagggtacctgtgcaggatgtgcaggtttgttacatgggtaaacgtgtgccatggtggtttgctgcacctatcaacccatcacctatgtattaagcccaacatgcattagctgtttttcctaatgctctccctcccctcaccccaccacccaacaagccctgtgtgtgtcgttcccctccctgtgtgtccatgtgttctcatttttcagctcccacttataagtgagaacatgtggtgtttggttttctgttcctgcgttagtttgctgaggataatggctgccagcttcatccatgtccttgcaaaggacatgatcttgtttctttttataactcagaaataagaccacccatctacaaccatctgatctttgacaaacctaacaaaaacaagcaatggggaaaggattccctatttaataaatggtcctgggagaactgactactcatatgcagaaaattgaaactggaccccttccttacaccttataaaaaaactaactcaagatagattaaagacttaaatgtaaaacacaaaactataaaaaccctagaagaaaatcaaggcaataccattcaaaacataagcacaggcaaaatcaccaaaagcaattgcagcaaaagcaaaaattgacaaatgggatctaattaaaccacattttccttatccattcatttgtcaatggacatttgggctgcctctccctcttggctattgtgaataataataggtgcaatgaacgtgggtgtacaaatatctctttaagattccgctttaattcttttggatatgtaccctgaactgggacataatcgtatggtgattatattttcaattttttgagaaatcactatattgttttccataatggttgcaccattttacattctctacaacagtgaacaagggttacaatttctctacatcctttccaatgcctgttattttctgttgtgatagtgtccatcctaatgagtatgaggtgatatttcattgtgctttttatttgcatttctcttatgattagtgatatggagcattttttcatatgcttgtgggtcatttgtatgtctttggagaattgtctatttaagcccttcacccattttttaataaggttattttgttgtaggagtttataatatattctggatattaaccctttatcatatatataatttaaaaatattgtctcccattctgtaggtcatctttttttgctgttgattatctcctttgatgcacagaccttaagtttgaaatcatctcatttgtcttctgttgttgttgtcgttgcctgtgtttttggtgtcatatctaagaaaccattgccaaataccctgaagctttttccttatgttttcttctaggagttttataggtttaggtcttatggttaggtctttaatgcattttgagttaatttttgtatatggcataaggtaagagtacaacctcattcttttcatgtagatatccagttttcctcacacaatttgttgaagaaactgtcctttattgtatagtcttggcacccttgtcaaagatcataaagagcatgtatagaaggctttatttctggactctgttccattggtctgtatgtctgcctttatgtcagtaccacaccatcttgattactgttgccttacagtatgtattgaaatcaagaagtgtgaagtctccaacattgttctttttcaggattactttggcaatttaaaatctcttgatcttatatgaattttagatttttttcctgtagaaattaccattgagattttgatagagatggcattgattctgtagattgttttgggtgttatagacagtatgacaatattaaattttccagtctataaacatgggatgtttcatcattgtgtttatatcatctttgatttattttggttatctcttaacaattttcattgtacaagtcttttgcctcctcagttcgattcattcctaagcattgtattctttctgatacttttgtaaatggggttgtttccttaatttccttttcagattagtcattgttagtttatggaaatataactcatttgtgtgttgattttgtatcctgcaaatttgctaacatcatttattagttttagtgggttttttgtgggatctttaggattttctgtatatatgagattatgtaatctgggaacagataattttacttcttttccatttgaatgccttttatttatttttcttgtctaattgctgtgactagagtttctagtactgtgttgaatagaaatggcaagaataggcatcctttccttgttcccattcttagaggagaaactttcagttttttgctattaagaatcattttaactgtggacttttcatatgtgacttttattatgtgtaggtactttccttccattcttcatttgttgagtgtttttatcatgaaaaagtgttgaattttgtcagatggttttgtgcatcaattgagatgatcatgtgtttttacttcttcattctgttaatgtggcatattatactgatagtttttcacatatggaaccttccttgcattcctggtataaatccaacttgttcatggtatataatctttttaatatgagaagttttgcatcagtatgcattagggatattggtctgtagttttcttgttgtgataatggatttacatttaaacactattggcgagtagctcctgagaaatgccatacattgtaaacatctaaaatggcttaaggactctataataaagttgttctatgtataatgtcacattaaaaaagtttggtgaaatttgctaaagagaattcagaattaaatacaaatagaaatatcttaaacatctccttttttattaaggatgtttaattccttgctgccaaaggaatgtcttctaggatgtggagaggaaattcttatacatacattcttatagaatgtaggagaaaaaaatgtcttcctctgtgcttgatctgatttctaacattctgagtttccatggttgattggttggttagttgcttgaaaaaggtatgtgtgtatgtcaaagaaaaatttgaacatttcaacacagttatctcaaatcatataatttaaactaaaatgggggcattagctataataatgttgaacctatagccccataaaggctatttctcaatactaacagactgttttggaagtctctcacacaactttaatacatattttacttcttggtacatttccttacattcaatagaaacataaaaattagttgaggctgggtgcggtggctcacacctgtaatcccagcactttgggaggccgaagtggacggataacgaagtcaggagatccagaccatcctggctaatgtggcaaaaccttgtctctactgaaaatacaattaggtgtggtggcacgtgcctgtagtcccagctactcacaaggctaaggcaggagaattgcttaaacccgggaggcagaggttgcagtgagccgagatcgcaccactgcactccagcctgggcgacagagcgagactttgtctcaaaaaaaaaaaaaaaaaattagttgaaattctgtctcagttatgaaaggtagctttattcttttggttttctattttgagttatgactgagaaataacctgtggcagaggttttcttaacaatgtattgtggccagtgcacctgtttcaagattactctaaacagaaacaagagtgaaataaatttgtggagttaataataaaaatgttattctctgtggtatgttttcttttcttttgtttttttaagacagggtcttgctctgttgcccaggctggagtgcagtggcgccatcttggctcactgcaacctctgcctcccaggttcaagcgattctcctgcctcaggcccctgagtagctgggattacaggcacgtaccaccacgcctagctaatttttatatttttagtagagacagggtttcaccatgttggtcatgttggtctcgaactcctgacctcatgatctgccccctgccagcctcccacagtgctgggattacaggcgtgagccaccgcgcccggcctgtattttttcatttcatagaatttccagataaaagtcagagtcaactagttttataatactgaaaagttttgcaaatacaagcatgtcacttaatgatggggatgtgttctgagaaatgtgtcattaggtgattccgtcattgtgcaaatgtcatagagtatacttacataaacctaggtggtatgtagcctatatgatatatgtggcttatatgatataccctattgctcctaggctgcaaacctgtatagcatgttactatcctgaagtcaatgggcaattgtaacacaatggcaagtatttgtgtattgaaacataagggtatagtaaaaatatggtactattaatacaactgtatgggactaccattgtatatgcggtccatcattggccaaactttctttatggtggcacaagactacataataaatggtaaagtaaccagacttggagatggggaggagggagaagaatgccaaatgtatgtttcctcccacagactagcaggcatctagtatcaggcaacacacttatacaatcatttcctactcctgtctcacacttgccagaatctggatttaattttttttttttattgttagaccagtaatgcagttttcatattttgtataacaaaaaagttgtaggacttttccttcttactgtgaaaatatgactcattaaatacataacttttagtattggccatttgtattcttatactcttagtcacaaagataactacgttttacaaagtcgaaaggttaataatagcagctagcatgaattcagagcttgctatgtattgagcactgggtttaaacacatatgctctattttattaagtccttatagtaggaaagtgaagaatacactagtcactgaagttccttcagtgctcaagactcaagtgcactatagcaagacaaacaccaggaaacatgtaactctgcttcgaatactttagcaagtgaatggcccagtttgggaaagcaagacaaacactggatccataatggctttttacacctgattggaatgcagaaaaaatggaaccactaactactaatattatctttgccagcaggtgagcagtacatacctgacttcagaggattttattgaaaccaatagatgatggttaataaaaacaaatatcctcagctttccttcttttacttgtcatctgacaaccactatttagctaaaactaaaatggattagagatacaagatgatgaataacaattattaacttttgtgagcatataatgtgagtcagctactacactaagaactttacatagattcccttaattttctcaataatcctatgagaactgtccttgtatgagcaaccccattttatagctgaagatacttgaggcttcaagaggttatataaacatgttcatggtcaacagttattaagcagtgagtgacattctcacgagattaatgagatcatctgtgtgagatacaaatacatctattgtgtatgtttgtgtttttaatggagaccaatggtatgacagaatgaagtccttttatcagtcagtcaagatctgatgtggctattcaaatataggcaaaactgatgttttccttttttagttaagttaaatgtaatatttaaataactgtgaatatttagaatattcacagaattgtgcaaccatcaccacaatctttagaatattttcatcatctcaaaaagatactttgtagcctttagcacccttcccactgtacccccccccatacccctggccataggcaactactaaattactttctgtccctgcaggttgtcctattctggatactttgtatatgtggaatcataaaataggtgatcttttatgactggcatctttcaagcatgtttttgatattttcatccatgttgtaggatattaaattaactttttgcataggaataactgagctagaaaagcattggctgaatatgtctgcagaatgtagcatttagaaaagaaagacttccaggagacaaatgaggtagtaactaaaatgtaaataactaaaaattagctttgagtctttaataaactacctttcatgttttgctttcactaggaatgaacttttaattttttggagctataaatacatgtagtttctaaagacatctttgtttttaaagaaaagacatctgcaggctttttcttagtgttcctattaagtatgtatgagtgtgtgcatttgtgtgtgtgtgtgtgcatgtttagtgactttacatgtgtatacatacaaatgtacacagtcatatatgactagtggtacatggaatctcaatttaattgtaaaagtgaaggagttctaaaactagttttagtatagtaagtgggatatagtaagaatgtcctacaatttaaaagcttatttggttatcttcaatggattttgaatgtattcaatactagatttgcgagtgactatgcctttttagtaaacaaaagaaaggctataatgtagaaagtatatggaataagcataacatctgtcctaatctaattttcaggcacatctccagacttcaatttggctgaaataattcatgccacggacctgtgcacatgcctggaattgagagacacagttaaaagactccaagttgctttctgccttttgaaaactcctgaaaaccatccctttggactctggaattctacacagctcaaccaagactttgcttgaatgtttacattttctgctcgctgtcctacatatcacaatatagtgttcacgttttgttaaaactttggggtgtcaggagttgagcttgctcagcaagccagcatggctaggatgagctttgttatagcagcttgccaattggtgctgggcctactaatgacttcattaaccgagtcttccatacagaatagtgagtgtccacaactttgcgtatgtgaaattcgtccctggtttaccccacagtcaacttacagagaagccaccactgttgattgcaatgacctccgcttaacaaggattcccagtaacctctctagtgacacacaagtgcttctcttacagagcaataacatcgcaaagactgtggatgagctgcagcagcttttcaacttgactgaactagatttctcccaaaacaactttactaacattaaggaggtcgggctggcaaacctaacccagctcacaacgctgcatttggaggaaaatcagattaccgagatgactgattactgtctacaagacctcagcaaccttcaagaactctacatcaaccacaaccaaattagcactatttctgctcatgcttttgcaggcttaaaaaatctattaaggctccacctgaactccaacaaattgaaagttattgatagtcgctggtttgattctacacccaacctggaaattctcatgatcggagaaaaccctgtgattggaattctggatatgaacttcaaacccctcgcaaatttgagaagcttagttttggcaggaatgtatctcactgatattcctggaaatgctttggtgggtctggatagccttgagagcctgtctttttatgataacaaactggttaaagtccctcaacttgccctgcaaaaagttccaaatttgaaattcttagacctcaacaaaaaccccattcacaaaatccaagaaggggacttcaaaaatatgcttcggttaaaagaactgggaatcaacaatatgggcgagctcgtttctgtcgaccgctatgccctggataacttgcctgaactcacaaagctggaagccaccaataaccctaaactctcttacatccaccgcttggctttccgaagtgtccctgctctggaaagcttgatgctgaacaacaatgccttgaatgccatttaccaaaagacagtcgaatccctccccaatctgcgtgagatcagtatccatagcaatcccctcaggtgtgactgtgtgatccactggattaactccaacaaaaccaacatccgcttcatggagcccctgtccatgttctgtgccatgccgcccgaatataaagggcaccaggtgaaggaagttttaatccaggattcgagtgaacagtgcctcccaatgatatctcacgacagcttcccaaatcgtttaaacgtggatatcggcacgacggttttcctagactgtcgagccatggctgagccagaacctgaaatttactgggtcactcccattggaaataagataactgtggaaaccctttcagataaatacaagctaagtagcgaaggtaccttggaaatatctaacatacaaattgaagactcaggaagatacacatgtgttgcccagaatgtccaaggggcagacactcgggtggcaacaattaaggttaatgggacccttctggatggtacccaggtgctaaaaatatacgtcaagcagacagaatcccattccatcttagtgtcctggaaagttaattccaatgtcatgacgtcaaacttaaaatggtcgtctgccaccatgaagattgataaccctcacataacatatactgccagggtcccagtcgatgtccatgaatacaacctaacgcatctgcagccttccacagattatgaagtgtgtctcacagtgtccaatattcatcagcagactcaaaagtcatgcgtaaatgtcacaaccaaaaatgccgccttcgcagtggacatctctgatcaagaaaccagtacagcccttgctgcagtaatggggtctatgtttgccgtcattagccttgcgtccattgctgtgtactttgccaaaagatttaagagaaaaaactaccaccactcattaaaaaagtatatgcaaaaaacctcttcaatcccactaaatgagctgtacccaccactcattaacctctgggaaggtgacagcgagaaagacaaagatggttctgcagacaccaagccaacccaggtcgacacatccagaagctattacatgtggtaactcagaggatattttgcttctggtagtaaggagcacaaagacgtttttgctttattctgcaaaagtgaacaagttgaagacttttgtatttttgactttgctagtttgtggcagagtggagaggacgggtggatatttcaaatttttttagtatagcgtatcgcaagggtttgacacggctgccagcgactctaggcttccagtctgtgtttggtttttattcttatcattattatgattgttattatattattattttattttagttgttgtgctaaactcaataatgctgttctaactacagtgctcaataaaatgattaatgacaggatggggttcccctgtgcttttaccagtagcatgaccccttctgaagccatccgtagaaagtactttgtcctccaaaaagctaacatacggttttgaagcagcattgaaacttttgtagcaatctggtctatagacttttaactcaagaagctaaggctagacttgttaccttcgttgaatgatgttagttgactgtactgtaatgttgtatcaactgaattgaatgtttgcctttaaacaatgaattttctttttctttccttttttttttttttgttgtaatagttaaagaggcttagaacaagctaacaggcaatagaaatatgtatatcagattttttaatgtaacaaactacatgttaattgttatcttattctttttatctttagtagacacttttaaaagaaaagacaagtttgttgtgtttaactcaccaacacgtggtgtataatgaagacagaactataataaattagttttgttctgattttttagaacacttgcaataatgtatcatttatagttcttgctagttgcagtggtaatatttttcacatccataaaaacaactaccaaaataaatcagctgtagcatgttgctttttaaagctaggccctaaaaggttttaattctttttctaagggaagaaatgtctattttaattaagatattttaatgaacaggatttctgtattttaaatagtactgactagcacctaatgggcagtgggagggtggttcatatgaagaaaaaaaggtgtattgttgtatcccatgcataaataaaggtaaatatatatatacaccaatatattcatatatactcacacacatcccaacctgtcacacacaatgcgtgtgtatatatatatgaatatattggatatgtcatttctgtaagagttttgttaaaacctgattttcttttgtagtatccacattcttcatcaaagtacaaaaacgtctgtggagtgtcacaaactgtatgacatgttatttctttttaacagttgtctatatgcttagacccgtgttagtctctatatctgtgtggcaatatctgctgagacaagtaaatgattaatagaaaacaaaacaacttccgtacagttcaaacttttcatccaaatatatatagacagttttggagaattgtttcaagattatagaggaaacatgtaacatttagagcagatggaactaggtttaggtagaaggccagttccacaaagggcagagggagggatgggatttaataggtaaagaagaacccatttgaaaataaaggttgtttcaaaaggcagctgccgccaggcacacagcattccatcagacaggtgccagacaagcaaaagcaaggaagttggcagaaagaaagtccaggtgatgtaggttgaggtatttctttctttggcagtatcttgcttttgtgaatcactttattaaaaaaaatcactttcttcccattcttaaggggttttggcaaacagaatttcagatcttgaaacaaatgagctgcaacagaaaaataagtacctgagctcgaggtatcccttcccaaagccttcacttattggtgagaaaaacctgggcccagggagggcttgcagcttacccagttcggactcctgccagttcagcgctctgcactccattgattgttctaggcccggcagcctgttaggttcctgtgcagggcccctcttttgcagttctgaataacctctagtgcccctgcttcaaaaatgcagtacatcctcaagttcccatttactcaggatacattttagcacagggcagatcagacggtgtctctactgttagtattgcaaagtatgtatgggaaacacagagaattggagctgcgttgaatgcaaacttgaggtgtttcccttgaggaattcttgtcttcaaacgtctgcagagtaatggaccatgttacaactttcctgttcatctgtgaaccatgaaaatggatggcactgatgcattagaccctcagcagcctgcaattgcaaatctgcgaggtttcattcggcccataaagcaaacatttgaacttacacagaatgagcacttaaatacgggtgcaataaatgaagggaaaaacctcagccgtttctccattctgaagatatagcaagcaccgggaaatctaagatttttcatcaacaatatcttctgccagcccagtttggggggaaaaaccccttttacatttttcttcagtaaagtactggaacttacttttccctgtctgtgctaatgagctgattttcagctgatagaaaacaaaatgatagagtacttttttccttggccaagtattttctcatttgtatttaatttcataaattagacagccagtgaaattagacctcaaactaggtcctgatggataatgaatgttatgtcacctttaacagtgaagtggttattataggtcactttctaatttcatattttcccttttgctttctgctgccttcagggtatatagtgtatctctaacctgatttttcaaggttatttttggagcagtttcttaaaacaggcattccctaacttgctcatttaattaatgaaaaattgaactgatgccatggatataaaaacaaatgtaatgtttgattgtcagtgtttctgatttggcaaaaaggaatcatctctatttttttgcaaacaatatcaaagtgcatattttctctca

SEQ ID NO: 22 - DNA, 37 bp, Artificial Sequence
  a forward primer
gatcggatccatggctaggatgagctttgttatagca

SEQ ID NO: 23 - DNA, 39 bp, Artificial Sequence
  a reverse primer
gatcctcgagttaccacatgtaatagcttctggatgtgt